(19) United States
(12) Patent Application Publication
(10) Pub. No.: US 2026/0048101 A1
(43) Pub. Date: Feb. 19, 2026

(54) GLP-1R ANTAGONISM TO IMPROVE NUTRITION FOLLOWING GASTROINTESTINAL SURGERY

(71) Applicants: Amylyx Pharmaceuticals, Inc., Cambridge, MA (US); The Board of Trustees of the Leland Stanford Junior University, Stanford, CA (US)

(72) Inventors: Colleen M. Craig, Burlingame, CA (US); Tracey L. McLaughlin, Santa Clara, CA (US)

(21) Appl. No.: 19/358,715

(22) Filed: Oct. 15, 2025

Related U.S. Application Data
(63) Continuation of application No. PCT/US2024/025627, filed on Apr. 22, 2024.
(60) Provisional application No. 63/497,502, filed on Apr. 21, 2023.

(51) Int. Cl. A61K 38/26; A61K 9/00; A61K 47/12; A61K 47/26; A61P 3/08

(52) U.S. Cl. CPC A61K 38/26; A61K 9/0019; A61K 47/12; A61K 47/26; A61P 3/08

(57) ABSTRACT

Methods of improving nutrition in subjects, including individuals who have undergone gastrointestinal surgery, by avexitide therapy are disclosed. Avexitide may be administered subcutaneously or intravenously as a part of a liquid pharmaceutical formulation. Avexitide therapy may improve weight and/or nutritional intake/status in subjects.

### CROSS-REFERENCE TO RELATED APPLICATIONS

This application is a continuation of and claims benefit under 35 U.S.C. § 120 from PCT Application No. PCT/US2024/025627, filed on Apr. 22, 2024, which claims priority to U.S. Provisional Application No. 63/497,502, filed on Apr. 21, 2023. The entire contents of each of these earlier applications are incorporated herein by reference.

### BACKGROUND

Patients who undergo gastrointestinal (GI) surgery (gastrectomy, esophagectomy, others) for treatment or prevention of malignancies of the upper GI tract commonly experience unwanted weight loss in the early postoperative period. This affects over 15% of total gastrectomy patients and over 35% of esophagectomy patients. Body weight loss and/or low body mass index (BMI) is associated with poorer responses to chemotherapy, recurrence, and decreased survival. Thus, it is of extreme importance to prevent body weight loss following upper GI surgery for patients with cancer. Low BMI is also associated with increased mortality in non-cancer patients and thus the benefits of improved nutrition in underweight individuals may extend to non-cancer patients as well.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are, among others, the following non-limiting exemplary embodiments. Including among the embodiments of the present invention are methods of improving nutrition in a subject, comprising a step of administering to the subject a pharmaceutical formulation comprising avexitide, wherein the subject has undergone at least one gastrointestinal surgery. In some embodiments of the above methods, administering the pharmaceutical formulation improves nutrition in the subject in comparison to nutrition in the subject prior to administering of the pharmaceutical formulation. In some embodiments of the above methods, administering of the pharmaceutical formulation increases nutritional intake in the subject in comparison to nutritional intake in the subject prior to administering of the pharmaceutical formulation. In some embodiments of the above methods, administering of the pharmaceutical formulation increases appetite in the subject in comparison to appetite in the subject prior to administering of the pharmaceutical formulation. In some embodiments of the above methods, administering of the pharmaceutical formulation increases food tolerance in the subject in comparison to food tolerance in the subject prior to administering of the pharmaceutical formulation. In some embodiments of the above methods, administering of the pharmaceutical formulation decreases food and/or taste aversion in the subject in comparison to food and/or aversion in the subject prior to administering of the pharmaceutical formulation. In some embodiments of the above methods, administering of the pharmaceutical formulation decreases food intolerance in the subject in comparison to food intolerance in the subject prior to administering of the pharmaceutical formulation. In some embodiments of the above methods, administering of the pharmaceutical formulation decreases eating avoidance in the subject in comparison to eating avoidance in the subject prior to administering of the pharmaceutical formulation. In some embodiments of the above methods, of the pharmaceutical formulation increases caloric intake in the subject in comparison to caloric intake in the subject prior to administering of the pharmaceutical formulation. In some embodiments of the above methods, administering of the pharmaceutical formulation increases feeding behavior in the subject in comparison to feeding behavior in the subject prior to administering of the pharmaceutical formulation. In some embodiments of the above methods, administering of the pharmaceutical formulation decreases fear of eating in the subject in comparison to fear of eating in the subject prior to administering of the pharmaceutical formulation. In some embodiments of the above methods, administering of the pharmaceutical formulation decreases eating-related worries in the subject in comparison to eating-related worries prior to administering of the pharmaceutical formulation. In some embodiments of the above methods, administering of the pharmaceutical formulation decreases one or more gastrointestinal symptoms in the subject in comparison to the one or more one or more gastrointestinal symptoms in the subject prior to administering of the pharmaceutical formulation. In some embodiments of the above methods, the subject experiences an increase in weight or a decrease in rate of weight loss upon administering of the pharmaceutical formulation. In some embodiments of the above methods, the subject experiences dietary liberalization upon administering of the pharmaceutical formulation. In some embodiments of the above methods, administering of the pharmaceutical formulation decreases the rate of hypoglycemia events upon administering of the pharmaceutical formulation.

Also included among the embodiments of the present invention are methods of improving nutritional intake in a subject in need of improved nutritional intake, comprising administering to the subject a pharmaceutical formulation comprising avexitide, wherein the subject has undergone at least one gastrointestinal surgery. Also included among the embodiments of the present invention are methods of improving appetite in a subject in need of appetite improvement, comprising administering to the subject a pharmaceutical formulation comprising avexitide, wherein the subject has undergone at least one gastrointestinal surgery. Also included among the embodiments of the present invention are methods of improving food tolerance in a subject in need of food tolerance improvement, comprising administering to the subject a pharmaceutical formulation comprising avexitide, wherein the subject has undergone at least one gastrointestinal surgery. Also included among the embodiments of the present invention are methods of decreasing food and/or taste aversion in a subject in need of decreased food and/or taste aversion, comprising administering to the subject a pharmaceutical formulation comprising avexitide, wherein the subject has undergone at least one gastrointestinal surgery. Also included among the embodiments of the present invention are methods of decreasing food intolerance in a subject in need of decreased food intolerance, comprising administering to the subject a pharmaceutical formulation comprising avexitide, wherein the subject has undergone at least one gastrointestinal surgery. Also included among the embodiments of the present invention are methods of decreasing food avoidance in a subject in need of decreased food avoidance, comprising administering to the subject a pharmaceutical formulation comprising avexitide, wherein the subject has undergone at least one gastrointestinal surgery. Also included among the embodiments of the present invention are methods of increasing caloric intake in a subject in need of increase in caloric intake, comprising administering to the subject a pharmaceutical formulation comprising avexitide, wherein the subject has undergone at least one gastrointestinal surgery. Also included among the embodiments of the present invention are methods of increasing feeding behavior in a subject in need of increase in feeding behavior, comprising administering to the subject a pharmaceutical formulation comprising avexitide, wherein the subject has undergone at least one gastrointestinal surgery. Also included among the embodiments of the present invention are methods of decreasing fear of eating in a subject in need of decrease in fear of eating, comprising administering to the subject a pharmaceutical formulation comprising avexitide, wherein the subject has undergone at least one gastrointestinal surgery. Also included among the embodiments of the present invention are methods of decreasing eating-related worries in a subject in need of decrease in eating-related worries, comprising administering to the subject a pharmaceutical formulation comprising avexitide, wherein the subject has undergone at least one gastrointestinal surgery. Also included among the embodiments of the present invention are methods of decreasing one or more gastrointestinal symptoms in a subject in need of decrease in one or more gastrointestinal symptoms, comprising administering to the subject a pharmaceutical formulation comprising avexitide, wherein the subject has undergone at least one gastrointestinal surgery. Also included among the embodiments of the present invention are methods of increasing weight in a subject in need of weight increase, comprising administering to the subject a pharmaceutical formulation comprising avexitide, wherein the subject has undergone at least one gastrointestinal surgery. Also included among the embodiments of the present invention are methods of decreasing the rate of weight loss in a subject in need of decrease rate of weight loss, comprising administering to the subject a pharmaceutical formulation comprising avexitide, wherein the subject has undergone at least one gastrointestinal surgery. Also included among the embodiments of the present invention are methods of preventing weight loss in a subject in need of weight loss prevention, comprising administering to the subject a pharmaceutical formulation comprising avexitide, wherein the subject has undergone at least one gastrointestinal surgery. Also included among the embodiments of the present invention are methods of liberalizing diet of a subject in need of dietary liberalization, comprising administering to the subject a pharmaceutical formulation comprising avexitide, wherein the subject has undergone at least one gastrointestinal surgery. Also included among the embodiments of the present invention are methods of decreasing rate of hypoglycemia events in a subject in need of decreased rate of hypoglycemia events, comprising administering to the subject a pharmaceutical formulation comprising avexitide, wherein the subject has undergone at least one gastrointestinal surgery.

In some embodiments of the methods of the present invention, a few non-limiting examples of which are summarized above, administering of the pharmaceutical formulation decreases the rate of hypoglycemia events in a subject upon administering of the pharmaceutical formulation. In some embodiments of the methods of the present invention, prior to administering of the pharmaceutical formulation, a subject has elevated levels of glucagon-like peptide 1 (GLP 1) in comparison to a reference population level. In some embodiments of the methods of the present invention, a subject has been diagnosed prior to the at least one gastric surgery with one or more of gastric cancer, esophageal cancer, gastroesophageal reflux, increased risk of gastric cancer, or increased risk of esophageal cancer. In some non-limiting examples, at least one gastric surgery is gastrectomy, esophagectomy, or Nissen fundoplication. In some non-limiting examples, at least one gastric surgery is at least one bariatric surgery, such as, but not limited to, Roux-en-Y Gastric Bypass (RYGB), Vertical Sleeve Gastrectomy (VSG), or BPD. In some embodiments of the methods of the present invention, a subject has type 2 diabetes.

In some embodiments of the methods of the present invention, the pharmaceutical formulation administered to a subject comprises avexitide at a concentration of between about 2-225 mg/mL. In some embodiments of the methods of the present invention, the pharmaceutical formulation is a buffered liquid formulation. In some embodiments of the methods of the present invention, the pharmaceutical formulation is administered subcutaneously. In some embodiments of the methods of the present invention, the pharmaceutical formulation is administered to achieve a total daily dose of avexitide from about 40 mg to about 120 mg. In some embodiments of the methods of the present invention, the pharmaceutical formulation is administered to achieve a total daily dose of avexitide of about 90 mg. In some embodiments of the methods of the present invention, the pharmaceutical formulation is administered to achieve a total daily dose of avexitide from about 0.4 mg/kg to about 3 mg/kg. In some embodiments of the methods of the present invention, the pharmaceutical formulation is administered once daily (QD) or twice daily (BID). In some embodiments of the methods of the present invention, the pharmaceutical formulation is administered once daily (QD) at a dose from about 20 mg to about 120 mg, or about 90 mg. In some embodiments of the methods of the present invention, the pharmaceutical formulation is administered once daily (QD) at a dose of about 90 mg. In some embodiments of the methods of the present invention, the pharmaceutical formulation is administered twice daily (BID) at a dose from about 40 mg to about 60 mg. In some embodiments of the methods of the present invention, the pharmaceutical formulation is administered twice daily (BID) at a dose of about 45 mg. The embodiments summarized in this section and other embodiments of the disclosure are described in detail below.

### BRIEF DESCRIPTION OF THE DRAWINGS

The present disclosure includes the following figures. The figures are intended to illustrate certain embodiments and/or features of the embodiments of the present invention, and to supplement any description(s) of the embodiments of the present invention. The figures do not limit the scope of the embodiments of the present invention, unless the written description expressly indicates that such is the case.

FIG. 1 is a schematic illustration of a design of a Phase 2 7-day clinical evaluation study (Study A) (according to the present disclosure) of avexitide for improved eating behavior in cancer patients with total gastrectomy or esophagectomy. The clinical assessments performed were Buffet Test, MMTT, and Surveys (VAS, SHS, CNAQ, GSRS, HFS-II, SF-36). The 10d outpatient assessments were Dexcom pro blinded CGM and Chronometer.

FIG. 2 is a schematic illustration of a design of Phase 2 28-day clinical evaluation study (Study B) (according to the present disclosure) of avexitide for improved nutritional status and prevention of unwanted weight loss in cancer patients with total gastrectomy or esophagectomy. The clinical assessments performed were Anthropomorphic Measurements; Body composition by BIA, DEXA, or CT; Buffet Test with VAS and CNAQ; MMT with EHSS; and Surveys (SHS, GSRS, HFS-II, SF-36). The 10d outpatient assessments were Dexcom pro blinded CGM and Chronometer.

FIG. 3 is a schematic illustration of a design of Phase 2 28-day clinical evaluation study (Study C) (according to the present disclosure) of avexitide therapy in patients with severe hypoglycemia after gastrointestinal surgery, including Roux-en-Y Gastric Bypass (RYGB), vertical sleeve gastrectomy (VSG), esophagectomy, gastrectomy, and Nissen fundoplication. Patients were treated with avexitide at 90 mg QD for two weeks followed by 45 mg BID for two weeks or 45 mg BID for two weeks followed by 90 mg QD for two weeks. Each study arm had 8 patients. The assessments at baseline, end of Period 1, and End of Period 2 were Anthropomorphics (Wt, Ht, BMI)*, Surveys (SHS*, GSRS*, HFS-II*, SF-36, EQ-5D), and Labs. *Assessment pertains only to post-gastrectomy or esophagectomy patients.

FIG. 4 is a bar graph illustrating selected results-percent time in hypoglycemia—of a Phase 2 28-day clinical evaluation study (Study C) (according to the present disclosure) of avexitide therapy in patients with severe hypoglycemia after gastrointestinal surgery, including RYGB, VSG, esophagectomy, gastrectomy, and Nissen fundoplication. Percent time in hypoglycemia spent by the study participants is plotted on the y-axis. The bars are as follows: black—baseline; white—avexitide administration 45 mg BID (twice daily); grey—avexitide administration 90 mg BID.

FIG. 5 is a bar graph illustrating selected results—the number of hypoglycemia events in 14 days (“event rate”)—of a Phase 2 28-day clinical evaluation study (according to the present disclosure) of avexitide therapy in patients with severe hypoglycemia after gastrointestinal surgery, including RYGB, VSG, esophagectomy, gastrectomy, and Nissen fundoplication. The number of hypoglycemia events registered in the study participants in 14 days is plotted on the y-axis. The bars are as follows: black—baseline; white—avexitide administration 45 mg BID; grey—avexitide administration 90 mg BID. Percent Time in hypoglycemia was defined as total hours of continuous glucose monitoring (CGM) readings below 70 mg/L or 54 mg/dL divided by total CGM wear time. Event rate was defined as number of episodes below glucose range (70 or 54 mg/dL) for at least 15 minutes during each treatment period normalized to 14 days.

FIG. 6 is a bar graph illustrating selected results—number of diurnal hypoglycemia events in 14 days (“diurnal event rate”)—of a Phase 2 28-day clinical evaluation study (according to the present disclosure) of avexitide therapy in patients with severe hypoglycemia after gastrointestinal surgery, including RYGB, VSG, esophagectomy, gastrectomy, and Nissen fundoplication. The number of number of diurnal hypoglycemia events registered in the study participants in 14 days is plotted on the y-axis. The bars are as follows: black—baseline; white—avexitide administration 45 mg BID; grey—avexitide administration 90 mg BID. Event rate was defined as number of episodes below glucose range (70 or 54 mg/dL) for at least 15 minutes during each treatment period normalized to 14 days. Diurnal was defined as an event occurring starting from 8 am and up to 10 pm.

FIG. 7 is a bar graph illustrating selected results—percent time in hypoglycemia at CGM values of <70 mg/dL—of a Phase 2 28-day clinical evaluation study (according to the present disclosure) of avexitide therapy in patients with severe hypoglycemia after RYGB, VSG, and gastrectomy (as indicated). Percent time in hypoglycemia spent by the study participants is plotted on the y-axis. The bars are as follows: black—baseline; white—avexitide administration 45 mg BID; grey—avexitide administration 90 mg BID.

FIG. 8 is a bar graph illustrating selected results—percent time in hypoglycemia at CGM values of <54 mg/dL—of a Phase 2 28-day clinical evaluation study (according to the present disclosure) of avexitide therapy in patients with severe hypoglycemia after RYGB, VSG, and gastrectomy (as indicated). Percent time in hypoglycemia spent by the study participants is plotted on the y-axis. The bars are as follows: black—baseline; white—avexitide administration 45 mg BID; grey—avexitide administration 90 mg BID.

FIG. 9 is a bar graph illustrating selected results-percent time in hypoglycemia at CGM values of <40 mg/dL—of a Phase 2 28-day clinical evaluation study (according to the present disclosure) of avexitide therapy in patients with severe hypoglycemia after RYGB, VSG, and gastrectomy (as indicated). Percent time in hypoglycemia spent by the study participants is plotted on the y-axis. The bars are as follows: black—baseline; white—avexitide administration 45 mg BID; grey—avexitide administration 90 mg BID.

FIG. 10 is a line plot illustrating selected results—body weight of male animals—of an animal study according to the present disclosure. Body weight in g is plotted on the x axis. The plots are for four different groups of animals administered different avexitide dose levels as described in in Example 5 of the present disclosure.

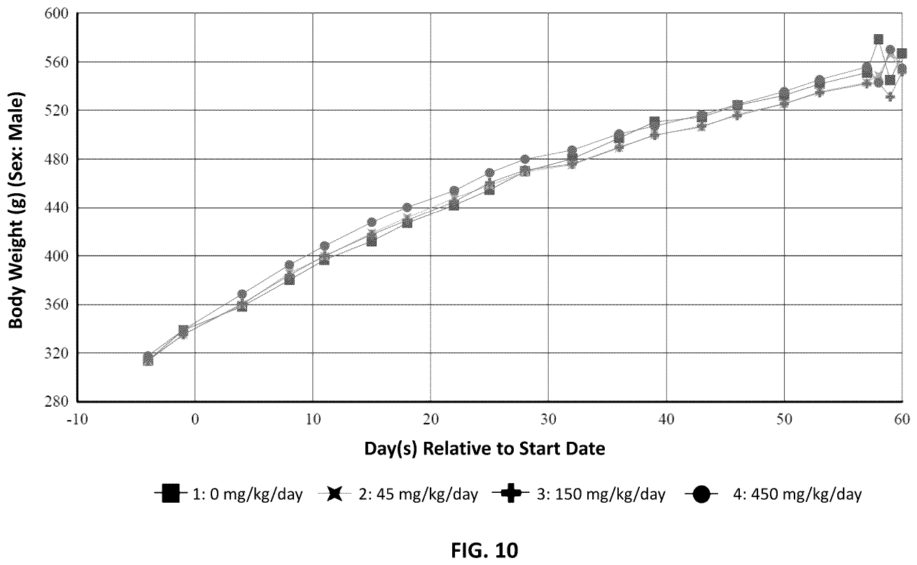

FIG. 11 is a line plot illustrating selected results—body weight of female animals—of an animal study according to the present disclosure. Body weight in g is plotted on the x axis. The plots are for four different groups of animals administered different avexitide dose levels as described in in Example 5 of the present disclosure.

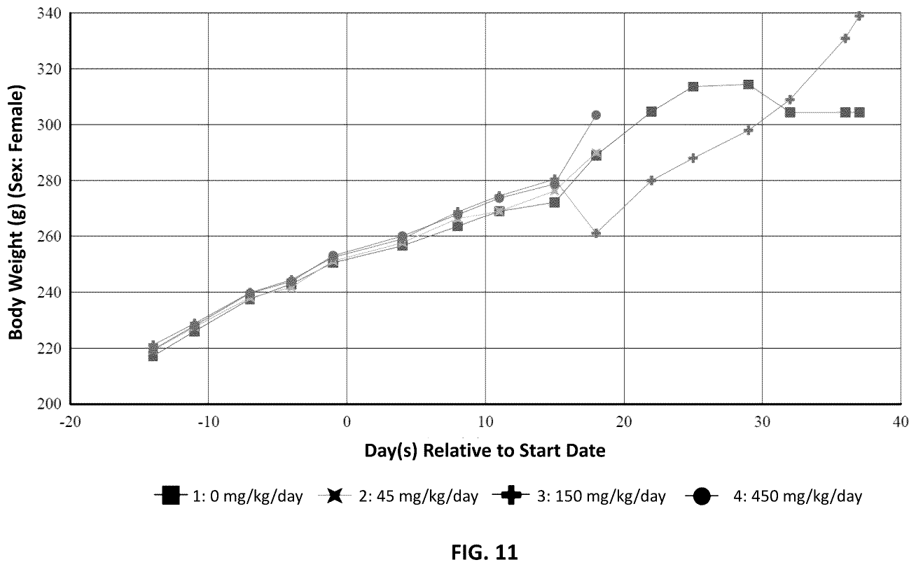

### DETAILED DESCRIPTION

### Overview

Described in the present disclosure are methods of improving nutrition in subjects by administering to the subjects a pharmaceutical formulation comprising a GLP-1 antagonist, for example avexitide. Individuals who undergo gastrointestinal (GI) surgery, such as, but not limited to, gastrectomy, esophagectomy, etc., which in some cases is performed for treatment or prevention of malignancies of the upper gastrointestinal tract, may experience rapid unwanted weight loss in the postoperative period, especially early in the early postoperative period. Among patients who are being treated for cancer, body weight loss and/or low body mass index (BMI) is associated with poorer responses to chemotherapy, recurrence, and decreased survival. Unwanted or excessive weight loss can also afflict those individuals who have had upper GI surgeries for indications other than malignancies, including, but not limited to, Nissen fundoplication and bariatric surgery, such as Roux-en-Y gastric bypass (RYGB) and vertical sleeve gastrectomy (VSG). While low body weight in these individuals has not necessarily been linked to increased mortality, malnutrition related to inadequate calorie, protein, or carbohydrate intake can cause muscle wasting, fatigue, muscle weakness/exercise intolerance, and mood changes such as depression. The reasons for weight loss are not fully elucidated. For example, postprandial GI symptoms (such as, but not limited to, nausea, early satiety, heart burn, dyspepsia, or bloating) in post-GI surgery patients are common. Also, post-GI surgery patients may experience slow gastric transit and/or gastric stasis (gastroparesis), which can be accompanied by emesis (vomiting). The patients may also experience reduced appetite and taste aversion, as well as hypoglycemia-related worries and fears. Some patients may have increased levels of glucagon-like peptide-1 (GLP-1), GIP, and/or PYY, which, among other things, promote insulin secretion, satiety, and/or inhibit gastric emptying, thereby decreasing food consumption. Any or all of the above factors may lead to food avoidance and decreased feeding. Such factors contribute to poor nutritional intake in post-GI surgery patients, resulting in weight loss, prevention of weight regain, and/or malnutrition. Among other things, the inventors conceived that targeted antagonism of the GLP-1 receptor with avexitide is useful as a treatment to improve nutrition in post-GI surgery patients.

The inventors unexpectedly discovered that avexitide administration in post-GI surgery patients can lead to improved nutrition in the patients, including, but not limited to, prevention of unwanted weight loss or improvement in weight regain in the early postoperative period, reduced gastrointestinal symptoms, dietary liberalization with improved nutrition/improved nutritional intake, improved appetite, and attitudes to feeding, reduced taste aversion, and reduced hypoglycemia fears and associated behaviors, including food avoidance. The inventors also made a surprising discovery that avexitide administration in post-GI surgery patients positively affected appetite (i.e. appetite increase was observed) and GI symptoms (i.e. decrease in GI symptoms was observed), the patients being a severely underweight population of post-GI surgery patients who were at high risk for malnourishment and therefore poor morbidity and mortality outcomes and/or responses to cancer treatment, such as chemotherapy. Thus, the inventors conceived that improved nutrition resulting from avexitide administration can improve the quality of life of the patients, improve their psychological well-being, increase body weight and/or prevent unwanted weight loss, reduce postprandial hypoglycemia, reduce GI symptoms, reduce need for prolonged gastrostomy tube feedings, reduce need for severe carbohydrate restriction, improve psychological well-being, and might lead to improved responses to chemotherapy and increased survival.

As compared to medical feeding procedures, such as gastrostomy tube and intravenous feeding, avexitide administration is less invasive, easy to self-administer, and has fewer potential side effects, such as increased risk of localized and systemic infections, malposition or blockage, and offers more independence and greater psychological acceptance. The examples provided herein demonstrate that subcutaneous administration of avexitide in individuals undergoing upper GI surgery for cancers prevents unwanted weight loss, possibly by mitigating the effects of supraphysiologic GLP-1 concentrations that result from surgery: central satiety, GI side effects due to altered motility and gastric accommodation, and severe reactive hypoglycemia. Accordingly, the inventors conceived that GLP-1 receptor antagonism administration, for example with the GLP-1 antagonist, avexitide, according to the methods of the present disclosure, should be incorporated into the multimodal approach to treatment of post-GI surgery patients.

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures, and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in describing the methods of the present invention are those well-known and commonly used. Known methods and techniques are generally performed according to conventional methods well-known and as described in various general and more specific references, unless otherwise indicated.

All numerical designations, for example, pH, temperature, time, concentration, and molecular weight, including ranges, are approximations which are varied (+) or (−) by increments of 0.1 or 1.0, as appropriate (for example, pH 5.4 or 5.5). It is to be understood, although not always explicitly stated, that all numerical designations are preceded by the term “about.” References to ranges include the endpoints unless indicated otherwise. For example, administration of avexitide at a concentration of 30 mg/mL to 180 mg/mL includes administration of 30 mg/mL or 180 mg/mL. Unless otherwise indicated, numerical ranges include all values and subranges tin the present disclosure, as if explicitly written out.

The terms “about” and “approximately,” as used in the present disclosure, shall generally mean an acceptable degree of error for the quantity measured, given the nature or precision of the measurements. Exemplary degrees of error are within 20%, 10%, 9%, 8%, 7%, 6%, 5%, 4%, 3%, 2%, or 1% of a given value or range of values. For example, any reference to “about X” or “approximately X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.01X. In another example, the terms “about” or “approximately” in relation to a reference numerical value can include a range of values plus or minus 10%, 9%, 8%, 7%, 6%, 5%, 4%, 3%, 2%, or 1% from that value. Thus, expressions “about X” or “approximately X” are intended to describe a claim limitation of, for example, “0.98X.” Numerical quantities given in the present disclosure are approximate unless stated otherwise, meaning that the term “about” or “approximately” can be inferred when not expressly stated. When the terms “about” or “approximately” are applied to the beginning of a numerical range, they apply to both ends of the range. Where a series of values is prefaced with the terms “about” or “approximately,” these terms are intended to modify each value included in the series.

As used in the present disclosure, the terms “a”, “an”, and “the” can refer to one or more unless specifically noted otherwise. For example, reference to “a compound” includes a plurality of compounds.

The use of the term “or” is used to mean “and/or,” unless explicitly indicated to refer to alternatives only, or the alternatives are mutually exclusive, although the disclosure supports a definition that refers to only alternatives and “and/or.” As used in the present disclosure “another” can mean at least a second or more.

As used in the present disclosure, and unless otherwise indicated, the terms “include,” “including,” and, in some instances, similar terms (such as “have” or “having”) mean “comprising.” The term “comprising” is intended to mean that the compounds, compositions and methods include the recited elements, but not excluding others. “Consisting essentially of” when used to define compounds, compositions and methods, means excluding other elements that would materially affect the basic and novel characteristics of the claimed invention. “Consisting of” means excluding any element, step, or ingredient not specified in the claim. Embodiments defined by each of these transition terms are within the scope of this invention.

“Avexitide,” which is also referred to as “exendin (9-39),” refers to a 31 amino acid peptide with an empirical formula of C₁₄₉H₂₃₄N₄₀O₄₇S and a molecular weight of 3369.8 Daltons. Avexitide comprises residues 9-39 of the GLP-1 receptor agonist exendin-4 and is a GLP-1 receptor antagonist with inverse agonist properties. See Montrose-Rafizadeh et al. (1997). The amino acid sequence for avexitide is shown as follows: H-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂ (SEQ ID NO: 1). Avexitide has a predicted isoelectric point of 4.69 and has a net charge of −1 at pH 6 that increases to a net charge of +4 at pH 3.0. As used herein, the term “avexitide” also encompasses pharmaceutically acceptable salts of avexitide (exendin (9-39)), including but not limited to sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate salts. In some embodiments, avexitide is in the form of an acetate or trifluoroacetate salt. Where not otherwise specified herein, avexitide acetate is used. Avexitide (exendin (9-39)) and pharmaceutically acceptable salts thereof are commercially available.

The terms “formulation” or “pharmaceutical formulation,” and other related terms and expression, as used herein, refer to a composition suitable for administration to a subject. A pharmaceutical formulation may be sterile, and preferably free of contaminants that are capable of eliciting an undesirable response within the subject. The compounds included in a pharmaceutical formulation are usually pharmaceutical grade. Pharmaceutical formulations can be designed for administration to subjects or patients in need thereof via a number of different routes of administration, including oral, intravenous, buccal, rectal, parenteral, intraperitoneal, intradermal, intramuscular, subcutaneous, inhalational and the like. In some embodiments, a pharmaceutical formulation as described herein is formulated for subcutaneously or intravenous administration.

As used herein, a “therapeutically effective amount” is an amount of an active ingredient (for example, avexitide or its pharmaceutically acceptable salt) that eliminates, ameliorates, alleviates, or provides relief of the symptoms or leads to clinical outcomes for which it is administered.

The terms “administer,” “administering,” and “administration” as used herein (and the related terms and expressions), refer to introducing a compound (for example, avexitide), a composition, or an agent into a subject or patient, such as a human. As used herein, the terms encompass both direct administration (for example, self-administration or administration to a patient by a medical professional) and indirect administration (for example, the act of prescribing a compound or composition to a subject).

“QD” and “BID” have their usual meanings of administration of a composition (for example, buffered liquid formulation of avexitide) once per day or twice per day, respectively. In some embodiments, administration once per day (QD) means that at least 20 hours, at least 22 hours, or about 24 hours elapse between administrations. In some embodiments, administration once per day means administration about every 24 hours. In some embodiments, administration twice per day (BID) means that at least 4 hours, at least 6 hours, at least 8 hours, at least 10 hours, at least 11 hours, or about 12 hours elapse between administrations. In some embodiments, administration twice per day means administration about every 12 hours.

As used herein, the terms “individual,’ “patient,” and “subject” interchangeably refer to an individual (for example, a human or a non-human mammal). The terms individual, subject, and patient, by themselves, do not denote a particular age, sex, race, or clinical status. The methods according to the present disclosure can be applied to any human race, including, for example, Caucasian (white), African American (black), Native American, Native Hawaiian, Hispanic, Latino, Asian, and European.

“Gastric emptying,” “stomach emptying,” and the related expressions refer to a process by which the context of the stomach moves out of the stomach. In a healthy subject, the contents of the stomach moves into duodenum for absorption int the small intestine. The rate or time of gastric emptying is the time it takes for food to empty out of the stomach, which can be measured by various appropriate tests, such as scintigraphy. For example, gastric emptying rate in a healthy human subject is about 1.5 to 2 hours, meaning the time it takes for stomach contents to move out of the stomach and into the small intestine. Delayed stomach emptying may be called “gastroparesis.” Gastric emptying is discussed, for example, in Maurer et al. (2021).

Glucagon-like peptide 1 receptor (GLP1R) a 7-transmembrane protein that functions as a receptor for glucagon-like peptide 1 (GLP-1) hormone, which stimulates glucose-induced insulin secretion. GLP1R becomes internalized in response to GLP-1 and GLP-1 analogs, and it plays an important role in the signaling cascades leading to insulin secretion. See, for example, Brubaker et al. (2002).

Glucagon-like peptide 1 (GLP-1) is an amino acid peptide hormone of 30 or 31 amino acids produced in the intestinal epithelial endocrine L-cells by differential processing of proglucagon, the gene for which is expressed in these cells. GLP-1, which is is an endogenous incretin hormone, is secreted by the L-cells of the distal small and large intestine in response to luminal nutrients. GLP-1, which is discussed in Holst (2007), is known have various physiological effects, including stimulation of insulin secretion, inhibitition of glucagon secretion, inhibition gastrointestinal motility, promotion of satiety. High GLP-1 concentrations alter gastrointestinal motility and gastric accommodation, which can promote anorexia, nausea, acid reflux, and bloating, thus potentially augmenting weight loss. Satiety is the result of several processes, including vagal nerve stimulation, gastric distension, and central nervous system effects. GLP-1 receptors are present in many parts of the brain, including areas implicated in the control of food intake and energy balance, and infusion into the brain of rats inhibits feeding activity, an effect that is blocked by co-infusion of exendin 9-39 (a GLP-1 antagonist) (Turton 1996). Data in rodents demonstrated that GLP-1 also promotes altered taste sensation, which possibly further promotes weight loss. In humans, administration of synthetic analogs of GLP-1 effectively induces weight loss of up to 15% of body weight. High GLP-1 concentrations can also promote postprandial insulin hypersecretion: this contributes to prolonged remission of diabetes in 85% of patients treated with RYGB. In a subset of patients, however, very high GLP-1 levels cause dysregulated insulin secretion and hypoglycemia (Salehi (2014)), which can be severe and frequent and lead to food avoidance, carbohydrate depletion, and weight loss. Similar to what has been observed following RYGB, GLP-1 concentrations may be elevated following other upper gastrointestinal surgeries. In particular, several studies show 3-5-fold elevations in GLP-1 levels following gastrectomy and esophagectomy, which are associated with dumping symptoms and hyperinsulinemic hypoglycemia. Thus, antagonism of GLP-1 in conditions in which unwanted weight loss is prevalent, particularly following upper gastrointestinal surgeries in which GLP-1 concentrations are high, is an attractive therapeutic possibility. The success of such treatment, however, is not obvious, as one prior study in which postbariatric patients were given an intravenous infusion of exendin 9-39 or saline for 240 min, followed by a free choice buffet meal test did not demonstrate increased food intake with administration of exendin 9-39 (Kittah et al. (2020)).

### Therapeutic Methods

Described in the present disclosure are methods of improving nutrition in a subject that involve administering to the subject a pharmaceutical formulation including avexitide (which may be referred to as “avexitide formulation”). Suitable pharmaceutical formulations are described elsewhere in the present disclosure. Methods of improving nutrition in a subject according to the present disclosure may be referred to as “therapeutic methods,” “avexitide therapy,” and by other related terms and expressions. When used according to the embodiments of the present invention, avexitide may exert its therapeutic effects by various mechanisms. Some of these mechanisms are discussed below but are not intended to limit any of the methods described in the present disclosure. It is to be understood that one or more mechanisms of avexitide action may be involved in the therapeutic methods described in the present disclosure.

For example, avexitide may improve nutrition in post-GI surgery patients by preventing taste aversion and GLP-1R-mediated satiety via central nervous system (CNS)-mediated mechanisms. The results of several animal studies support the notion that weight-loss/anorectic effects of GLP-1 are CNS-mediated. In a study reported in Sisley et al. (2014), GLP-1R knockout mice did not consume more food than wild-type mice, but also did not lose weight when administered liraglutide (GLP-1 agonist), which points to a CNS-mediated mechanism for the weight-loss/anorectic effects of GLP-1 agonism. As reported in Martin et al. (2009), dramatic reduction in sweet taste sensitivity, as well as enhanced sensitivity to umami-tasting stimuli, has been shown in GLP-1R knockout mice. The animal data reported in the above studies suggest that, although GLP-1 receptors are not necessary for normal food intake or body weight regulation, CNS GLP stimulation/agonism is necessary for the weight-loss/anorectic effects. Some studies showed that GLP-1R antagonism did not stimulate appetite, increase feeding behaviors, or cause weight gain. Melhorn et al. (2014) reported a study involving infusion of avexitide or placebo in normal-weight subjects, which showed that GLP-1R blockade failed to suppress satiety or increase feeding behavior in the subjects. Kulve et al. (2017) reported the effects of RYGB on CNS activation in response to visual and gustatory food cues, which may be mediated by central effects of GLP-1, but did not evaluate feeding behaviors in response to such visual and gustatory cues. However, a study described in Kittah et al. (2020) investigated GI symptoms and carbohydrate intake in post-GI surgery patients (RYGB patients and VSG patients) versus weight-matched nonsurgical controls in the presence and absence of avexitide and showed no changes in carbohydrate intake or the occurrence of GI symptoms in the surgical patients, whereas avexitide decreased carbohydrate consumption in non-surgical controls. In view of the above studies, the discovery by the inventors of avexitide therapy resulting in improved nutrition in post-GI surgery patients is surprising and unexpected.

Avexitide may improve nutrition in post-GI surgery patients by reducing GI symptoms, such as nausea, dyspepsia, heart burn, and early satiety (fullness). Avexitide may also improve nutrition in post-GI surgery patients by ameliorating the fears associated with food intake and/or reducing food avoidance. The reduction in GI symptoms may result, at least in part, from a parasympathetic nervous system-mediated mechanisms, for example, via the vagus nerve. The reduction in GI symptoms may also occur at least in part due to increased rate of nutrient transit through GI tract upon avexitide administration. It has been previously shown that avexitide administration after RYGB accelerated gastric emptying. In a study reported in Shah et al. (2014) avexitide administration significantly increased the rate of gastric emptying of the radiolabeled solid meal during the first 45 minutes after food ingestion in twelve RYGB patients but not in eight age-, sex-, and weight-matched controls. However, improved GI symptomatology in post-GI surgery patients upon administration of avexitide has not been reported. It is also possible that avexitide can improve nutritional intake in post-GI surgery patients by ameliorating hypoglycemia-related fears and resulting food avoidance. For example, fear of hypoglycemia is common among patients with insulin-dependent diabetes. Data (some of which is included in the present disclosure) exists that supports reduction in hypoglycemia-associated worries and behaviors upon administration of avexitide.

Embodiments of therapeutic methods according to the present disclosure may result in improved nutrition in a subject to whom avexitide therapy is administered. In the context of the present disclosure, the term “nutrition” broadly encompasses various aspects of nutritional intake and eating, which can be measured objectively and/or subjectively experienced. The term “nutrition” encompasses, but is not limited to, the process of consuming (taking in, consuming, or ingesting) nutrition by a subject, in particular, the process of eating, including chewing and swallowing, as well as different stages of utilizing nutrition, including various aspects of digestion, nutrient absorption, excretion, etc. The term “nutrition” also encompasses the results of consuming and utilizing nutrition, for example, but not limited to, nutritional intake by the subject as well as nutritional state of a subject. For example, the subject may have or be expected to have undesirable weight loss after undergoing GI surgery, as discussed elsewhere in the present disclosure. In another example, the subject may be in need of prevention of unwanted weight loss after undergoing GI surgery. In another example, the subject can be or can be expected to be malnourished (have or expected to have malnutrition, as discussed elsewhere in the present disclosure) due to disturbances in one or more aspects of nutrition. In another example, the subject may have or be expected to have malnourishment (discussed elsewhere in the present disclosure) post-GI surgery, with or without malnutrition. In one more example, the subject may have or may be expected to have cachexia and/or sarcopenia (both discussed elsewhere in the present disclosure) post-GI surgery. In yet another example, the subject may be in need of or desire to undergo dietary liberalization, as discussed elsewhere in the present disclosure.

In the context of the present disclosure, “nutritional intake” broadly encompasses composition of food consumed by a subject. In this context, food composition (which can be also referred to as “nutrient composition”) encompasses various aspects of food composition consumed by the subject, such as caloric content, macronutrient content (for example, the amounts of one or more carbohydrates, proteins, fats, fiber, fatty acids, cholesterol, amino acids, and water) consumed by the subject, and micronutrient (for example, the amounts of various vitamins and minerals) content consumed by the subject. “Improved nutritional intake” denotes an improvement in food composition consumed by the subjects. For example, improved nutritional intake is increased caloric content consumed by a subject, due to one or more of higher total number of calories consumed by the subject over a period of time (per meal, per day, per week, per month, etc.) or improved digestion and/or absorption of the food composition consumed.

In addition to nutritional intake, the term “nutrition,” as used in the present disclosure,” encompasses the subject's nutritional state as well as subjective experiences and attitudes related to food and eating. Accordingly, “improved nutrition” encompasses various improvements in the subject's nutritional state and improved subjective experiences, attitudes, and behaviors related to food and eating. Nutritional state of the subject may be based on objective indicators, such as weight, body mass index (BMI), body composition (such as fat and muscle content of a subject's body), blood levels of various macronutrients and micronutrients, etc.

In the context of the present disclosure, the term “nutrition” also encompasses subjective experiences, feelings, behaviors, and attitudes related to food and eating. Subjective feelings, experiences and attitudes may manifest in various ways, such as the feelings of taste (for example, whether the subject perceives food as tasting good or bad), appetite (which may be rated as “very poor,” “poor,” “average,” “good” or “excellent” or on a numerical scale), feelings of satiety during or after a meal and/or throughout of the day, feelings of hunger during or after a meal and/or throughout of the day, moods associated with food consumption (for example, mood during a meal), feelings of food aversion, fear of eating, anxiety associated with food consumption, etc. Subjective experiences may also encompass various GI symptoms and bodily reactions experienced before, during, or after food consumption. Such GI symptoms and bodily reactions may be physiological nature, psychosomatic, or both. For example, a subject may experience nausea and, in some cases, vomit, after swallowing food. In another example, a subject may experience dyspepsia (indigestion) after consuming food, including abdominal discomfort, described as burning sensation, bloating or gassiness, and/or nausea. In another example, a subject may experience heartburn after consuming food. In yet another example, a subject may experience satiety (fullness) very quickly after starting to eat. Subjective experiences, including various GI symptoms and bodily reactions, may be triggered by one or more specific foods, by all food, by the process of eating, or even by thinking of eating. GI symptoms and unpleasant bodily reactions may be triggered by specific foods and described as “food intolerance” with respect to these foods. Conversely, “food tolerance” is an ability to eat various foods without experiencing GI symptoms and unpleasant bodily reactions. Higher or lower food tolerance may describe how many foods (a spectrum of foods) are tolerated by a subject, and may also encompass amounts of specific food tolerated. For example, a subject capable of consuming higher amounts of dairy than another subject without experiencing GI symptoms and unpleasant bodily reactions may have higher dairy tolerance for dairy. Subjective experiences, feelings, and attitudes related to food and eating encompass food aversion, which is a very strong dislike of a particular food(s) or food generally. In case of food aversion, a sight, smell, taste or even a thought of food (generally or of specific food(s)) may cause a subject to experience feelings of disgust and, in some cases, unpleasant reactions, such as nausea. Higher or lower levels of food aversion may describe to how many foods (a spectrum of foods) a subject experiences aversion.

Subjective experiences, feelings, behaviors, and attitudes related to food and eating include eating avoidance, meaning that a subject may restrict their intake of food by amount and/or variety (avoidance of specific foods), resulting in nutritional deficiencies and negative health outcomes. Eating avoidance may be based on sensory aspects of the food, fear of GI symptoms and unpleasant reaction, lack of interest in food, or other factors. Higher or lower levels of eating avoidance may describe the level of subject's restriction on the amounts and/or variety of food consumed. Subjective experiences, feelings, behaviors, and attitudes related to food and eating include fear of eating, meaning that a subject is afraid of consuming food generally or specific foods. Fear of eating may manifest itself in physiological symptoms of fear and/or panic, such as increased heart rate, faster breathing or shortness of breath, sweating, trembling, etc. Higher or lower levels of fear of eating may describe the level (intensity) of subject's fear symptoms. Subjective experiences, feelings, behaviors, and attitudes related to food and eating also include eating-related worries, meaning that a subject worries (experiences anxiety) about consuming food generally or specific foods. Eating-related worries may stem from various sources. For example, a subject may be anxious about experiencing GI symptoms or unpleasant bodily reactions after eating. Higher or lower levels of fear of eating-related worries may describe the level (intensity) of subject's anxiety as well as the number of foods and situation provoking eating-related worries. An example of a behavior related to food and eating is eating behavior, which broadly encompasses meal timing, including meal frequency, quantity of food intake, food preferences and choices, etc. Higher or lower levels of eating behavior may encompass one or more of meal frequency, quantity of food consume, and a variety of food consumed. In one example, embodiments of therapeutic methods according to the present disclosure may result in improved or increased feeding behavior.

As discussed throughout the present disclosure, administering to a subject a pharmaceutical formulation including avexitide may lead to improvements in nutrition, including, but not limited to, nutritional intake, nutritional state, and attitudes, behaviors and subjective experiences related to food and eating. It is to be understood that improvements (which may be described as increases or decreases of various observable or subjectively experienced metrics and effects) upon administration of avexitide therapy are in comparison to various control values, such as in comparison to a corresponding control value in the same subject prior to the start avexitide therapy. For example, in some embodiments, treatment of a subject with avexitide therapy as described in the present disclosure improves nutrition in the subject in comparison to nutrition in the subject prior to administration of the avexitide therapy. In another example, in some embodiments, treatment of a subject with avexitide therapy as described in the present disclosure improves nutritional intake in the subject in comparison to nutritional intake in the subject prior to administration of the avexitide therapy. In yet another example, a subject treated with avexitide therapy as described in the present disclosure experiences an increase in weight or a decrease in rate of weight loss, in comparison to weight or a rate of weight loss, respectively, in the subject prior to administration of the avexitide therapy.

In some embodiments, treatment of a subject with avexitide therapy as described in the present disclosure results in one or more of the following effects: increase in appetite, increase in food tolerance, decrease in food intolerance, decrease in eating avoidance, decrease in food aversion, increase in caloric intake, increase in feeding behavior, dietary liberalization, decrease in fear of eating, or decrease in eating related-worries, including, but not limited to, hypoglycemia-related worries. Accordingly, some embodiments of the methods described in the present disclosure can be referred to as methods of increasing or improving appetite, methods of increasing or improving food tolerance, methods of decreasing food intolerance, methods of decreasing eating avoidance, methods of decreasing food aversion, methods of increasing caloric intake, methods of increasing feeding behavior, methods of dietary liberalization, methods of decreasing fear of eating, or methods of decreasing eating related-worries, including, but not limited to, hypoglycemia-related worries. In some embodiments, treatment of a subject with avexitide therapy as described in the present disclosure results in weight gain and/or prevention of post-surgical weight loss (for example, loss in excess of 10% presurgical body weight). Accordingly, some embodiments of the methods described in the present disclosure can be referred to as methods of affecting or increasing weight gain, methods of treating or preventing post-surgical weight loss, methods of treating or preventing undesirable weight loss, etc. In some embodiments, treatment of a subject with avexitide therapy as described in the present disclosure results in treatment or prevention of sarcopenia and/or cachexia, which can be measured by various physiological criteria, such as body composition (for instance, proportion of lean body mass, fat mass, water) or physical performance (for instance, amount of total activity and/or grip force). Accordingly, some embodiments of the methods described in the present disclosure can be referred to as methods of preventing or reducing sarcopenia or methods of preventing or reducing sarcopenia cachexia. In some embodiments, treatment of a subject with avexitide therapy as described in the present disclosure results in one or more of the following effects: improved taste and/or perception of one or more foods, improved mood during meals, after a meal, and/or throughout the day, reduced feeling of anxiety related to food and eating, or improved general sense of well-being. Accordingly, some embodiments of the methods described in the present disclosure can be referred to as methods of improving taste and/or perception of one or more foods, methods of improving mood during meals, after a meal, and/or throughout the day, methods of reducing of anxiety related to food and eating, or methods of improving well-being. In some embodiments, treatment of a subject with avexitide therapy as described in the present disclosure results in reduction of one or more GI symptoms, such as nausea, dyspepsia, or heartburn. Accordingly, some embodiments of the methods described in the present disclosure can be referred to as methods of reducing one or more GI symptoms. In some embodiments, treatment of a subject with avexitide therapy results in increased movement of food through the subject's GI tract (which, in some instances, may be referred to in the present disclosure as increase in gastric emptying). Accordingly, some embodiments of the methods described in the present disclosure can be referred to methods of improving or increasing the rate of movement of food through the GI tract or methods of increasing or improving gastric emptying.

In some embodiments, the effect of the treatment of subjects according to the methods of the present disclosure can be monitored and/o by various physiological measures. In one example, levels of various biomarkers are measured by various tests, such as blood tests. One example of a biomarker to measure nutritional status is blood albumin level. Physical performance can be measured by total activity and/or grip strength. In another example, weight or body mass index may be measured by various applicable procedures. For example, in order to monitor the magnitude of weight loss after a GI surgery, such as total gastrectomy or esophagectomy, weight or body mass index may be measured immediately following the GI surgery and at various time pints thereafter. The weight or body mass index measured immediately following the GI surgery as a point of reference would serve as a point of reference for monitoring weight loss. In another example, nutritional status can also be measured by quantification of the macronutrient composition of meals using various tools, such as diaries and computer/smartphone applications, some examples of which are discussed elsewhere in the present disclosure. Treatment of subjects according to the methods of the present disclosure may result in one or more of diet liberalization, increase in carbohydrate intake and improvements in macronutrient balance.

In another example, the reduction in the number (or rate) of hypoglycemia events, overall or, for example, diurnal (“diurnal” was defined as an event occurring, for example starting from 8 am and up to 10 pm) hypoglycemia events, may be measured, for example, as the number of diurnal hypoglycemia events occurring over a specific period of time. For instance, the time period may be from 1 to 20 days, such as one day, two days, three days, four days, five days, six days, seven days, eight days, nine days, ten days, eleven days, twelve days, thirteen days, sixteen days, seventeen days, eighteen days, nineteen days, or twenty days. The time period may also be more than 20 days, for example, 20-100 days, 20-50 days, 20-25 days, etc. A hypoglycemia event may be defined as number of episodes below a defined glucose level, for example, for example, 70 mg/L, 54 mg/dL, or 40 mg/dL. Percent time in hypoglycemia may be defined, for example, as total hours of continuous glucose monitoring (CGM) readings below a defined glucose level, for example, 70 mg/L, 54 mg/dL, or 40 mg/dL, divided by total CGM wear time. Event rate can also be measure by CGM and in this instance may be defined as number of episodes below a defined glucose level, for example, for example, 70 mg/L, 54 mg/dL, or 40 mg/dL, for at least 15 minutes during each time period. Treatment of subjects according to the methods of the present disclosure may result in at least about 10%, at least about 20%, at least about 30%, at least about 40%, at least about 30%, reduction in a rate of hypoglycemia events experiment by the treated subjects.

It is to be understood that changes (increase or decrease) related to the effects of avexitide therapy may be measured in comparison to various control values, for example, in comparison to a corresponding control value in the same subject prior to the start avexitide therapy. Other types of control values may also be used, such as a placebo control. In one example, a placebo control is used a cross-over design comparison (placebo vs. avexitide in an individual subject). In one more example, a placebo control is used in a parallel design comparison (placebo in one arm and avexitide in another arm of patients). For example, in some embodiments, treatment of a subject with avexitide therapy as described in the present disclosure increases appetite in the subject, in comparison to appetite in the subject prior to administration of the avexitide therapy. In one more example, in some embodiments, treatment of a subject with avexitide therapy as described in the present disclosure increases food tolerance in the subject, in comparison to food tolerance in the subject prior to administration of the avexitide therapy. In one more example, in some embodiments, treatment of a subject with avexitide therapy as described in the present disclosure decreases food aversion in the subject in comparison to food aversion in the subject prior to administration of the avexitide therapy. In one more example, in some embodiments, treatment of a subject with avexitide therapy as described in the present disclosure decreases food intolerance in the subject in comparison to food intolerance in the subject prior to administration of the avexitide therapy. In one more example, in some embodiments, treatment of a subject with avexitide therapy as described in the present disclosure decreases eating avoidance in the subject in comparison to eating avoidance in the subject prior to administration of the avexitide therapy. In one more example, in some embodiments, treatment of a subject with avexitide therapy as described in the present disclosure increases caloric intake in the subject in comparison to caloric intake in the subject prior to administration of the avexitide therapy. In one more example, in some embodiments, treatment of a subject with avexitide therapy as described in the present disclosure improves or increases feeding behavior in the subject in comparison to feeding behavior in the subject prior to administration of the avexitide therapy. In one more example, in some embodiments, treatment of a subject with avexitide therapy as described in the present disclosure decreases fear of eating in the subject in comparison to fear of eating in the subject prior to administration of the avexitide therapy. In one more example, in some embodiments, treatment of a subject with avexitide therapy as described in the present disclosure decreases eating-related worries in the subject in comparison to eating-related worries in the subject prior to administration of the avexitide therapy. In yet one more example, in some embodiments, treatment of a subject with avexitide therapy as described in the present disclosure decreases one or more GI symptoms in the subject in comparison to one or more GI symptoms in the subject prior to administration of the avexitide therapy.

Various subtypes of post-GI surgery subjects, including those discussed in the present disclosure, tend to experience similar and/or overlapping issues, such as, but not limited, to rapid emptying of food into the small intestine where GLP-1-secreting L-cells reside, causing exaggerated secretion of GLP-1, hypersecretion of insulin and resultant hypoglycemia. GLP-1 then may have direct effects on the CNS, impairing taste sensitivity and appetite, and direct effects on the GI system, slowing the rate of gastric transit, which causes satiety and nausea. Additionally, hypoglycemia (due to hyperinsulinemia) may cause autonomic symptoms (palpitations, diarrhea, sweating, shaking, nausea) and also neuroglycopenic symptoms (confusion, lethargy, altered behavior, incoordination, seizures, loss of consciousness, etc.). Treatment of the post-GI surgery subjects according to the methods of the present disclosure may be performed to treat one or more of various post-GI surgery issues, including, but not limited to those described in the present disclosure.

Effects of avexitide therapy according to the present disclosure may be referred to as “treatment effects” or “treatment endpoints” and measured by various methods. For example, nutritional intake of a subject may be registered and measured by entering the food consumed into a diary, a computer or a smartphone software, and calculating nutritional intake (manually or using software) based on known data on nutritional composition of various foods. Nutritional state of the subject may be assessed by measuring weight, body mass index (BMI), body composition (such as fat and muscle content of a subject's body), blood levels of various macro and micronutrients, etc. GI symptoms may be assessed using various diaries, questionnaires, and rating scales. Gastric emptying rate may be measured by suitable tests, such as scintigraphy. Subjective experiences and behaviors related to food and eating may be assessed using various diaries and questionaries. Some examples of questionnaires suitable for measuring some effects of avexitide therapy according to the present disclosure are Council on Nutrition and Appetite Questionnaire (CNAQ) (Table 1), Short Health Scale-GI (SHS-GI) questionnaire (Table 2), and Hypoglycemic Fear Survey-II (HSF-II) questionnaire (Table 3). The CNAQ is an 8-item questionnaire aimed at assessing appetite, which has been found to be valid in predicting clinically significant weight loss in older adults in community dwelling older adults and in long term care residents (Wilson et al. (2005)). Each item is scored on a 5-point scale with possible total score ranging from 8 (worst) to 40 (best). A score of ≤28 indicates significant risk of at least 5% weight loss within 6 months and with such patients classified as at risk of malnutrition as loss of appetite is a predictor for weight loss (Landi et al. (2010)). The answers to CNAQ are scored numerically (a=1, b=2, c=3, d=4, e=5), and the sum of the scores for all individual items constitutes CNAQ score. The SHS-GI is a 4-item self-administered questionnaire. Each item deals with one of the subjective health dimensions: symptoms, social function, disease-related worry and general well-being. Responses are graded on a 100-mm visual analog scale and maximum sum score for all items is 400, with higher values indicating worse outcome. The results, presented as an individual score for each of the four items, form a profile. Methods of measuring treatment effects and endpoints are also described elsewhere in the present disclosure, such as in the Examples section below. The HFS-II is a 33-item questionnaire with two subscales that measure 1) behaviors to avoid hypoglycemia and its negative consequences and 2) worries about hypoglycemia and its negative consequences.

TABLE 1
Council on Nutrition and Appetite Questionnaire (CNAQ).

1. My appetite is **
a. Very Poor
b. Poor
c. Average
d. Good
e. Very Good
2. When I eat **
a. I feel full after eating only a few mouthfuls
b. I feel full after about a third of a meal
c. I feel full about eating over half of a meal
d. I feel full after eating most of the meal
e. I hardly ever feel full
3. I feel hungry
a. Rarely
b. Occasionally
c. Some of the time
d. Most of the time
e. All of the time
4. Food Tastes **
a. Very bad
b. Bad
c. Average
d. Good
e. Very Good
5. Compared to when I was younger food tastes
a. Much worse
b. Worse
c. Just as a good
d. Better
e. Much Better
6. Normally I eat **
a. Less than one meal a day
b. One meal a day
c. Two meals a day
d. Three meals a day
e. More than three meals a day
7. I feel sick of nauseated when I eat
a. Most times
b. Often
c. Sometimes
d. Rarely
e. Never
8. Most of the time my mood is
a. Very Sad
b. Sad
c. Neither sad nor happy
d. Happy
e. Very happy
The answers are scored based on the following numerical scale: a = 1; b = 2; c = 3; d = 4; e = 5.
The sum of the scores for all individual items constitutes the CNAQ score. The sum of the scores for the individual items marked ** constitutes the SNAQ score.

TABLE 2
Short Health Scale-GI (SHS-GI) questionnaire.

1. Do you have symptoms/inconvenience from gastrointestinal tract?
No Symptoms Very Severe Symptoms
2. Are your daily life activities interfered by gastrointestinal symptoms/inconvenience?
Not at all Interferes to a very high degree
3. Do you have any worry caused by gastrointestinal symptoms/inconvenience?
No worry Constant worry
4. What is your general sense of well-being?
Very Well Dreadful
The answers are graded on a 10-mm visual analog scale and summarized for a sum score, with higher values indicating worse outcomes.

TABLE 3
Hypoglycemic Fear Survey-II (HSF-II) questionnaire.

| | |
|---|---|
| 1. | Eat snacks |
| 2. | Try to keep my BG above 70 mg/Dl |
| 3. | Measure my BG six or more times per day |
| 4. | Take someone with me when I go out |
| 5. | Limit my out-of-town travel |
| 6. | Limit driving |
| 7. | Avoid visiting friends |
| 8. | Stay home more than I would like |
| 9. | Limit my exercise/physical activity |
| 10. | Make sure others are around |
| 11 | Avoid sex |
| 12. | Keep my BG higher than usual in social situations |
| 13. | Keep my BG higher than usual when doing important tasks |
| 14. | Have other people check on me several times during the day or night |
| 15. | Carry glucose tabs or other high-sugar snacks |
| 16. | Not being aware that my BG is low |
| 17. | Not having food or snacks available |
| 18. | Passing out in public |
| 19. | Embarrassing myself in a social situation |
| 20. | Having a hypoglycemic episode when I am alone |
| 21. | Appearing drunk or stupid |
| 22. | Losing control |
| 23. | No one being around to help me during a hypoglycemic episode |
| 24. | Having a hypoglycemic episode while driving |
| 25. | Making mistakes or having accidents |
| 26. | Getting a bad evaluation or being criticized |
| 27. | Difficulty thinking clearly when responsible for others |
| 28. | Feeling light headed or dizzy |
| 29. | Accidentally injuring myself or others |
| 30. | Permanent injury or damage to my health or body |
| 31. | Low BG interfering with important things I am doing |
| 32. | Becoming hypoglycemic during sleep |
| 33. | Getting emotionally upset and difficult to deal with |
| 34. | Having seizures or loss of consciousness |

### Patient Population

In some embodiments, a subject to be treated according to the methods described in the present disclosure is a subject who has previously had a gastrointestinal (GI) surgery. In some embodiments, a subject has previously had a non-bariatric surgical procedure involving the gastrointestinal system, including, but not limited to, esophagectomy, for example, for treatment or prevention of esophageal cancer, Nissen fundoplication, for example for treatment of gastroesophageal reflux, or gastrectomy (such as for treatment or prevention or treatment of gastric cancer). In some non-limiting embodiments, a subject has previously had a bariatric or metabolic surgical procedure involving the gastrointestinal system, including, but not limited to, Roux-en-Y Gastric Bypass (RYGB), Vertical Sleeve Gastrectomy (VSG), placement of an endosleeve device, such as the EndoBarrier Gastrointestinal Liner System, also called an “endoluminal liner,” duodenal mucosal resurfacing (also referred to as duodenal ablation), biliopancreatic diversion (BPD), biliopancreatic diversion with duodenal switch (BPD-DS), partial bypass of the duodenum, involving duodeno-ileal or duodeno-jejunal anastomosis, vagal nerve blockade, and pyloroplasty. In some embodiments, a subject to be treated according to the methods described in the present disclosure may be referred to in the present disclosure as a “post gastrointestinal surgery” patient, “post-GI patient,” being “post-GI surgery,” a “post-GI surgery subject,” and by other related expressions. In some embodiments, the subject has undergone at least one gastric surgery. In some embodiments, the subject is treated immediately after undergoing surgery (for example, in cases when the surgery is not bariatric surgery). In some embodiments, the subject to be treated previously had a gastrointestinal surgery at least one year prior to the onset of treatment. In some embodiments, the subject is a human. In some embodiments, the subject is an adult. In some embodiments, the subject is a juvenile (that is, a post-GI surgery juvenile).

In some embodiments, the subject has undergone gastrectomy. In some embodiments, the subject experiences a post-gastrectomy syndrome, which is characterized, for example, by a lowered tolerance for large meals, rapid emptying of food into the small intestine or “dumping,” abdominal cramping pain, diarrhea, lightheadedness after eating as well as increased heart rate and sharp drops in blood sugar levels. In some embodiments, the subject has undergone esophagectomy. In some embodiments, the subject experiences a post-esophagectomy syndrome, which may include various symptoms, such as, but not limited to, dysphagia, reflux, delayed gastric emptying, dumping syndrome, weight loss, and chronic diarrhea.

In some embodiments, post-GI surgery, the subject experiences or is expected to experience various complications related to disturbances in nutrition. Some non-limiting examples of such complications are anemia as a result of vitamin B12, iron malabsorption, and osteoporosis. When vitamin B12 is poorly absorbed, anemia and, in some cases, poor nerve function can occur. Iron deficiency anemia may develop because removal of the stomach often leads to a marked decrease in the production of gastric acid, which is necessary to convert dietary iron to a form that is more readily absorbed in the duodenum. Osteoporosis develops as a result of poor calcium absorption. Following GI surgery, calcium absorption may become less efficient as a result of rapid emptying of the stomach.

Subjects to be treated according to the methods described in the present disclosure may be identified by any suitable method. For example, the subjects may have undesirable weight loss after undergoing GI surgery. Undesirable weight loss encompasses weight loss that is unintentional, and may also be referred to as involuntary or unintended weight loss. Unintentional weight loss does not encompass some types of weight loss that is an expected consequence of treatment (weight loss from diuretic therapy in patients with heart failure) or as a result of a known illness. However, some other types of weight loss that are expected as a consequence of treatment, such as weight loss after GI surgery, are still undesirable and are included in the scope of the expression “undesirable weight loss.” For example, the subject may be expected to have undesirable weight loss post-GI surgery, for example, due to the patient being underweight or of normal weight prior to undergoing GI surgery. Subjects to be treated according to the methods described in the present disclosure may have clinically important weight loss, which is generally defined as loss of more than 5 percent of usual body weight over 6 to 12 months.

Subjects to be treated according to the methods described in the present disclosure may have malnutrition. Various criteria may be used to establish the diagnosis of malnutrition. For example, the following criteria for the diagnosis of malnutrition have been recommended in a consensus statement from the Academy of Nutrition and Dietetics (Academy) and the American Society for Parenteral and Enteral Nutrition (ASPEN) in 2012. Two or more of the following six characteristics are to be present for diagnosis of malnutrition: insufficient energy intake; weight loss; loss of muscle mass; loss of subcutaneous fat; localized or generalized fluid accumulation that may mask weight loss; diminished functional status as measured by handgrip strength. In another example, the malnutrition criteria were introduced in 2018 from the Global Leadership Initiative on Malnutrition (GLIM). The GLIM was established in order to develop a global consensus on the identification and diagnostic criteria for malnutrition to facilitate comparison of malnutrition prevalence, treatment, and outcomes. The above 2018 criteria include an appreciation of the role of acute and chronic inflammation, which is not represented in the International Classifications of Diseases 10th revision (ICD-10) codes. According to the GLIM, the diagnosis of malnutrition requires the combination of at least one phenotype criterion and one etiologic criterion: phenotype criteria—non-volitional weight loss, low body mass index (BMI), or reduced muscle mass; etiologic criteria—reduced food intake or absorption, or underlying inflammation due to acute disease/injury or chronic disease. In one more example, clinically accepted definition of malnutrition includes unintentional weight loss (>5% in 3 months, or >10% of indefinite time) as a component of one set of diagnostic criteria. After making the diagnosis of malnutrition, a more comprehensive assessment of nutritional status can be performed by dietitians or nutritionists, who can develop individualized care and treatment plans.

Subjects to be treated according to the methods described in the present disclosure may have malnourishment (not getting sufficient quantities of required nutrients) post-GI surgery, with or without malnutrition. Malnourishment may or may not involve weight loss. Subjects to be treated according to the methods described in the present disclosure may be expected to have malnourishment, which may or may not involve weight loss, post-GI surgery. Treating such subject according to the methods described in the present disclosure may prevent malnourishment. Subjects to be treated according to the methods described in the present disclosure may have or expected to have cachexia and/or sarcopenia post-GI surgery. In the latter situation, treating such subject according to the methods described in the present disclosure may prevent cachexia and/or sarcopenia. Cachexia is generally defined as weight loss from loss of muscle mass (with or without fat loss). Sarcopenia is characterized by loss of muscle mass, strength, and performance.

In some embodiments, subjects to be treated according to the methods described in the present disclosure experience food aversion, eating avoidance, fear of feeding, and other related behaviors and/or psychological issues. In some embodiments, subjects to be treated according to the methods described in the present disclosure experience one or more of reduced appetite, food aversion or taste aversion. In some embodiments, subjects to be treated according to the methods described in the present disclosure experience postprandial GI symptoms, such as, but not limited to, nausea, early satiety, heart burn, dyspepsia, or bloating. In some embodiments, subjects to be treated according to the methods described in the present disclosure experience slow gastric transit and/or gastric stasis (gastroparesis, which can be accompanied by emesis (vomiting). Gastroparesis is a syndrome of objectively delayed gastric emptying in the absence of a mechanical obstruction and cardinal symptoms of nausea, vomiting, early satiety, belching, bloating, and/or upper abdominal pain. Gastroparesis can be categorized by extent of delay in gastric transit (mild through severe). In some embodiments, subjects to be treated according to the methods described in the present disclosure experience hypoglycemia-related worries. In some embodiments, subjects to be treated according to the methods described in the present disclosure must follow strict or restrictive diets, involving frequent small, low glycemic index meals.

In some embodiments, subjects to be treated according to the methods described in the present disclosure are in need of dietary liberalization. Post GI surgery, subjects typically have to be extremely restrictive with their diets, such as having to take in frequent small low-glycemic index meals and avoid carbohydrates by and large. In the context of the present disclosure, dietary liberalization can mean an ability for the subjects to be less restrictive and an ability to introduce carbohydrates into the diet, resulting in a more balanced macronutrient composition.

In some embodiments, subjects to be treated according to the methods described in the present disclosure have elevated GLP-1 levels prior or after GI surgery. GLP-1 levels may be measured by various methods, some of which are discussed in Holst (2007). The levels of GLP-1 are elevated in comparison to a reference population level. In humans, fasting systemic plasma concentrations of “total” GLP-1 (including metabolites generated by DPP-4-mediated degradation) are typically in the range of 5-10 pmol/L and can increase up to 40 pmol/L in response to a meal (Müller et al. (2019)).

### Dosing Regimens

In some embodiments, avexitide, in the form of a suitable pharmaceutical formulation (which can be referred to as an “avexitide formulation” in the present disclosure), is administered (for example, subcutaneously administered) at a total daily dose in the range of about 40 mg to about 120 mg, for example, about 50 mg to about 120 mg a day, about 60 mg to about 120 mg a day, about 70 mg to about 120 mg a day, about 80 mg to about 120 mg a day, about 90 mg to about 120 mg a day, about 50 mg to about 110 mg a day, about 60 mg to about 110 mg a day, about 70 mg to about 110 mg a day, about 80 mg to about 110 mg a day, about 90 mg to about 110 mg a day, about 50 mg to about 100 mg a day, about 60 mg to about 100 mg a day, about 70 mg to about 100 mg a day, about 80 mg to about 100 mg a day, or about 90 mg to about 100 mg a day. For example, a total daily dose of avexitide may be from about 40 mg to about 90 mg from about 40 mg to about 75 mg, from about 40 mg to about 60 mg, from about 50 mg to about 90 mg, from about 45 mg to about 75 mg, from about 60 mg to about 90 mg, or from about 60 mg to about 75 mg. In some embodiments, avexitide is administered at a total daily dose of at least about 40 mg, for example, at least about 45 mg, at least about 50 mg, at least about 60 mg, at least about 75 mg, or at least about 90 mg. In some embodiments, avexitide is administered at a total daily dose of about 40 mg, about 45 mg, about 50 mg, about 55 mg, about 60 mg, about 65 mg, about 70 mg, about 75 mg, about 80 mg, about 85 mg, about 90 mg, or about 120 mg. In some embodiments, avexitide is administered at a total daily dose of 85 mg or 95 mg.

In some embodiments of the methods according to the present disclosure, the subjects can have a body weight of less than about 60 kg, less than about 59 kg, less than about 58 kg, less than about 57 kg, less than about 56 kg, less than about 55 kg, less than about 54 kg, less than about 53 kg, less than about 52 kg, less than about 51 kg, or less than about 50 kg, and such subjects can receive a dose avexitide from about 0.4 mg/kg/day to about 3 mg/kg/day. For example, a subject may receive a dose of avexitide of about 0.4 mg/kg/day, about 0.75 mg/kg/day, about 1 mg/kg/day, about 1.125 mg/kg/day, about 1.2 mg/kg/day, about 1.5 mg/kg/day, about 2 mg/kg/day, or about 3 mg/kg/day. In some embodiments subjects can receive about 0.375 mg/kg/dose or about 0.75 mg/kg/day. In some embodiments, subjects can receive about 0.56 mg/kg/dose or about 1.125 mg/kg/day. In some embodiments of the methods according to the present disclosure, the subjects can have a body weight of less than about 60 kg, less than about 59 kg, less than about 58 kg, less than about 57 kg, less than about 56 kg, less than about 55 kg, less than about 54 kg, less than about 53 kg, less than about 52 kg, less than about 51 kg, or less than about 50 kg, and can receive a dose avexitide of about 0.4 mg/kg/day to about 3 mg/kg/day. In the above embodiments, avexitide can be administered BID (twice daily) or TID (thrice daily).

In some embodiments, a therapeutically effective amount of avexitide (or a pharmaceutically acceptable salt thereof) is administered once daily (“QD”). QD administration is well-known in the medical arts. In some embodiments, QD doses are administered (for example, subcutaneously administered) at about 24-hour intervals (for example, 7 a.m. on successive days). However, shorter (for example, about 8 a.m. and about 6 a.m. on successive days) or longer (for example, about 7 a.m. and about 9 a.m. on successive days) intervals between administration are possible provided the administrations are at least about 18 hours apart. Preferably, the administrations are at least about 18 hours, about 19 hours, about 20 hours, about 21 hours, about 22 hours, about 23 hours, or 2 about 4 hours apart. Preferably, the administrations are not more than about 30 hours apart. Methods of timing the administration of QD dosing are described, for example, in PCT Patent Applications No. PCT/US2016/033837 and No. PCT/US/2019/0562778. In some instances, the dosage administered can occur more frequently (for example, two times a day) or less frequently (for example, once every other day).

In some embodiments, the avexitide formulation is administered (for example, subcutaneously or intravenously administered) twice daily/twice per day (“BID”). BID administration is well known in the medical arts. The formulation can be administered at specific points in the day or schedule of a subject, for example, morning, afternoon, evening, night, before or during or after meals, before bedtime, etc. In some embodiments, the formulation is administered about once every 12 hours. In some embodiments BID doses are administered (for example, self-administered) at about 12-hour intervals (for example, about 7 a.m. and about 7 p.m.). However, shorter (for example, about 8 a.m. and about 6 p.m.) or longer (for example, about 7 a.m. and about 10 p.m.) intervals between administrations are possible. In some embodiments, the administrations are at least about 4 hours, about 6 hours, about 7 hours, about 8 hours, about 9 hours, about 10 hours or about 11 hours apart. Preferably the administrations are not more than about 15 hours apart. Methods of timing the administration of BID dosing are described, for example, in PCT Patent Applications No. PCT/US2016/033837 and No. PCT/US/2019/0562778. In some embodiments, the avexitide formulation is administered (for example, subcutaneously or intravenously administered) thrice daily/thrice per day (“TID”).

Dosing of avexitide in adults may be from about 40 mg QD to about 120 mg QD. For example, dozing of avexitide in adults may be from about 40 mg QD to about 100 mg QD, from about 40 mg QD to about 75 mg QD, from about 40 mg QD to about 60 mg QD, from about 50 mg QD to about 90 mg QD, from about 45 mg QD to about 75 mg QD, from about 60 mg QD to about 100 mg QD, from about 60 mg QD to about 75 mg QD, or from about 85 mg QD to about 95 mg QD. Dosing of avexitide in adults may be from about 20 mg BID to about 60 mg BID. For example, dozing of avexitide in adults may be from about 20 mg BID to about 55 mg BID, from about 20 mg BID to about 50 mg BID, from about 20 mg BID to about 45 mg BID, from about 25 mg BID to about 50 mg BID, from about 25 mg BID to about 47 mg BID, from about 37 mg BID to about 47 mg BID, or from about 40 mg BID to about 47 mg BID.

In some embodiments, the avexitide formulation is administered (for example, subcutaneously or intravenously administered) at a dose of from about 20-65 mg BID or from 25-50 mg BID. In some embodiments, the avexitide formulation is administered at a dose of about 20 mg BID. In some embodiments, the avexitide formulation is administered at a dose of about 25 mg BID. In some embodiments, the avexitide formulation is administered at a dose of about 30 mg BID. In some embodiments, the avexitide formulation is administered at a dose of about 35 mg BID. In some embodiments, the avexitide formulation is administered at a dose of about 40 mg BID. In some embodiments, the avexitide formulation is administered at a dose of about 45 mg BID. In some embodiments, the avexitide formulation is administered at a dose of about 50 mg BID. In some embodiments, the avexitide formulation is administered at a dose of about 55 mg BID. In some embodiments, the avexitide formulation is administered at a dose of about 60 mg BID. In some embodiments, the avexitide formulation is administered at a dose of about 65 mg BID. In some embodiments, a formulation comprising avexitide at a concentration of about 30 mg/ml or higher is administered at a dose of from about 20 to about 65 mg BID, for example, at a dose of about 45 mg BID.

In some embodiments, the avexitide formulation is administered (for example, subcutaneously or intravenously administered) at a total daily dose of from about 40 to about 120 mg, or from about 60 to about 100 mg, or from about 85 to about 95 mg, and is administered BID. In some embodiments, the avexitide formulation is administered at a total daily dose of about 90 mg and is administered BID (i.e., about 45 mg BID). In some embodiments, the avexitide formulation is administered at a total daily dose of about 100 mg and is administered BID (i.e., about 50 mg BID). In some embodiments, the avexitide formulation is administered at a total daily dose of about 80 mg and is administered BID (i.e., about 40 mg BID); the avexitide formulation is administered at a total daily dose of about 120 mg and is administered BID (i.e., about 60 mg BID)

In some embodiments, the avexitide formulation is administered (for example, subcutaneously or intravenously administered) at a dose of from about 40 to about 120 mg QD, for example, from about 60 to about 100 mg QD or from about 85 to about 95 mg QD. In some embodiments, the avexitide formulation is administered at a dose of about 40 mg QD. In some embodiments, the avexitide formulation is administered at a dose of about 50 mg QD. In some embodiments, the avexitide formulation is administered at a dose of about 60 mg QD. In some embodiments, the avexitide formulation is administered at a dose of about 70 mg QD. In some embodiments, the avexitide formulation is administered at a dose of about 80 mg QD. In some embodiments, the avexitide formulation is administered at a dose of about 90 mg QD. In some embodiments, the avexitide formulation is administered at a dose of about 100 mg QD. In some embodiments, the avexitide formulation is administered at a dose of about 120 mg QD.

In some embodiments, the avexitide formulation is administered (for example, subcutaneously or intravenously administered) at a total daily dose of from about 60 to about 120 mg and is administered QD. In some embodiments, the avexitide formulation is administered at a total daily dose of about 60 mg and is administered QD (i.e., about 60 mg QD). In some embodiments, the avexitide formulation is administered at a total daily dose of about 70 mg and is administered QD (i.e., about 70 mg QD). In some embodiments, the avexitide formulation is administered at a total daily dose of about 75 mg and is administered QD (i.e., about 75 mg QD). In some embodiments, the avexitide formulation is administered at a total daily dose of about 80 mg and is administered QD (i.e., about 80 mg QD). In some embodiments, the avexitide formulation is administered at a total daily dose of about 90 mg and is administered QD (i.e., about 90 mg QD). In some embodiments, the avexitide formulation is administered at a total daily dose of about 120 mg and is administered QD (i.e., about 120 mg QD).

In some embodiments, the avexitide formulation is administered (for example, subcutaneously or intravenously administered) at a total daily dose of from about 60 to about 120 mg and is administered BID or QD. In some embodiments, the avexitide formulation is administered at a total daily dose of about 60 mg and is administered QD (i.e., about 60 mg QD) or BID (i.e., about 30 mg BID). In some embodiments, the avexitide formulation is administered at a total daily dose of about 70 mg and is administered QD (i.e., about 70 mg QD) or BID (i.e., about 35 mg BID). In some embodiments, the avexitide formulation is administered at a total daily dose of about 80 mg and is administered QD (i.e., about 80 mg QD) or BID (i.e., about 40 mg BID). In some embodiments, the avexitide formulation is administered at a total daily dose of about 90 mg and is administered QD (i.e., about 90 mg QD) or BID (i.e., about 45 mg BID). In some embodiments, the avexitide formulation is administered at a total daily dose of 120 mg and is administered QD (i.e., about 120 mg QD) or BID (i.e., about 60 mg BID).

In some embodiments, the avexitide formulation is administered (for example, subcutaneously or intravenously administered) twice daily (BID) within about 60 minutes prior to morning and evening meals (or prior to the two main meals of the day). In some embodiments, the avexitide formulation is administered at least about 60 minutes prior to a meal (for example, at least about 60 minutes prior to a morning meal and/or at least about 60 minutes prior to an evening meal). In some embodiments, the administrations prior to the morning and evening meals (or prior to the two main meals of the day) are at least about 6 hours apart. In some embodiments, the administration of the formulation is not timed to meals. In some embodiments, the avexitide formulation is administered (for example, subcutaneously or intravenously administered) once daily (QD) in the morning or at night to maximally cover the morning and evening meals. For example, in some embodiments, the formulation is administered at night after the evening meal or early in the morning prior to the morning meal (for example, at least about 60 minutes prior to the morning meal).

The dosages and dosage ranges described above are exemplary adult doses, and may vary depending upon the age and weight of a subject, as would be known by those skilled in the pharmaceutical arts. It will be appreciated that in some embodiments, dosage may be increased or decreased during the course of treatment. For example, some physicians may desire to treat with a low or initiating (starting) dose, escalate to an increased dose if the initiating dose does not provide sufficient therapeutic benefit, and maintain the initiating dose if the initiating dose provides sufficient therapeutic benefit.

### Routes of Administration

In some embodiments, a pharmaceutical formulation comprising avexitide (for example, a buffered liquid formulation as described elsewhere in the present disclosure) is administered to a subject by subcutaneous administration. Subcutaneous administration includes subcutaneous injections and subcutaneous infusions. Subcutaneous injection involves using a syringe and a short needle to inject a liquid pharmaceutical formulation in the fatty tissue under the skin. Subcutaneous infusion, or hypodermoclysis, is a subcutaneous technique in which liquids are infused into the subcutaneous space via small-gauge needles. Subcutaneous infusions may be performed by infusion pump or manually (for example, through a syringe). Sites of injection or infusion, include, but are not limited to, injection in the thigh, abdomen, upper arm region, back, or upper buttock region. In some embodiments, avexitide is injected using an insuflon. In some embodiments, a composition comprising avexitide (for example, a buffered liquid formulation as disclosed elsewhere in the present disclosure) is formulated for subcutaneous administration. In one embodiment, the avexitide composition is formulated for subcutaneous administration according to a once daily (QD), twice daily (BID), or thrice daily (TID) regime. In some embodiments, a pharmaceutical formulation comprising avexitide (for example, a buffered liquid formulation as described elsewhere in the present disclosure) is administered to a subject by intravenous administration, either as a bolus or by intravenous infusion over time. Intravenous administration can be conducted using a syringe or a catheter. Administration of a pharmaceutical formulation comprising avexitide encompasses self-administration.

### Avexitide Formulations

In some embodiments, the therapeutic methods disclosed herein comprise formulation including avexitide or a pharmaceutically acceptable salt thereof (“avexitide formulation”). In some embodiments, the formulation comprises avexitide at a concentration of at least 30 mg/mL. In some embodiments, the pharmaceutical formulation is a buffered liquid formulation. Pharmaceutical formulation comprising avexitide are described, for example, in PCT Patent Application No. PCT/US2016/033837 and No. PCT/US/2019/0562778

In some embodiments, the formulation comprises avexitide at a concentration of about 2-250 mg/mL, about 2-200 mg/mL, about 2-180 mg/mL, about 2-90 mg/mL, about 2-20 mg/mL, about 2-29 mg/ml, about 1-20 mg/ml, about 30-250 mg/mL, about 30-225 mg/mL, about 30-200 mg/mL, about 30-180 mg/mL, about 30-150 mg/mL, about 30-120 mg/mL, about 30-90 mg/mL, about 30-60 mg/mL, about 30-70 mg/mL, about 40-250 mg/mL, about 40-225 mg/mL, about 40-200 mg/mL, about 40-180 mg/mL, about 40-120 mg/mL, about 45-90 mg/mL, about 45-75 mg/mL, about 60-250 mg/mL, about 60-225 mg/mL, about 60-200 mg/mL, about 60-180 mg/mL, about 60-120 mg/mL, about 60-90 mg/mL, about 90-250 mg/mL, about 90-225 mg/mL, about 90-200 mg/mL, about 90-180 mg/mL, about 90-120 mg/mL, about 30-70 mg/mL, about (for example, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 200 mg/mL, about 220 mg/mL, or about 220 mg/mL). In some embodiments, the formulation comprises avexitide at a concentration of about 30 mg/ml. In some embodiments, the formulation comprises avexitide at a concentration in the range of about 40 mg/mL to about 50 mg/mL. In some embodiments, the formulation comprises avexitide at a concentration of about 45 mg/mL. In some embodiments, the formulation comprises avexitide at a concentration in the range of about 30 mg/mL to about 60 mg/mL. In some embodiments, the formulation comprises avexitide at a concentration in the range of about 30 mg/mL to about 90 mg/mL. In some embodiments, the formulation comprises avexitide at a concentration in the range of about 45 mg/mL to about 90 mg/mL. In some embodiments, the formulation comprises avexitide at a concentration of about 60 mg/mL. In some embodiments, the formulation comprises avexitide at a concentration of about 75 mg/mL. In some embodiments, the formulation comprises avexitide at a concentration of about 90 mg/mL. In some embodiments, the formulation comprises avexitide at a concentration of about 120 mg/mL. In some embodiments, the formulation comprises avexitide at a concentration of about 175 mg/mL. In some embodiments, the formulation comprises avexitide at a concentration of about 200 mg/mL.

In some embodiments, the formulation comprises avexitide or a pharmaceutically acceptable salt thereof in a physiologically acceptable buffer having a pH in the range of about 5 to about 6. In some embodiments, the buffer is compatible with subcutaneously or intravenous administration. In some embodiments, the physiologically acceptable buffer is a buffer that results in a liquid formulation having a pH at or about physiological pH, or within a relatively narrow pH range near physiological pH (for example, between about 5.0 to about 8.0). In one embodiment, the buffered liquid formulation comprises a physiologically acceptable buffer having a pH above 5.0 and up to about 6. In some embodiments, the physiologically acceptable buffer has a pH above 5.0 and up to about 5.5. In one embodiment, the physiologically acceptable buffer has a pH in the range of 5.2 to 5.8 (for example, 5.2, 5.3, 5.4, 5.5, 5.6, 5.7, or 5.8). In one embodiment, the physiologically acceptable buffer has a pH in the range of 5.0 to 5.5 (for example, 5.1, 5.2, 5.3, 5.4, or 5.5). In one embodiment, the physiologically acceptable buffer has a pH in the range of about 5.5 to about 6 (for example, 5.5, 5.6, 5.7, 5.8, 5.9, or 6). In one embodiment, the physiologically acceptable buffer has a pH of about 5.5. In one embodiment, the physiologically acceptable buffer has a pH of about 5.6. In one embodiment, the physiologically acceptable buffer has a pH of about 5.7. In one embodiment, the physiologically acceptable buffer has a pH of about 5.8.

In one embodiment, the physiologically acceptable buffer comprises an acetate buffer, a citrate buffer, a phosphate buffer, a histidine buffer, or a mixture thereof. In one embodiment, the physiologically acceptable buffer comprises sodium acetate, potassium acetate, trisodium citrate, magnesium citrate, potassium citrate, potassium phosphate, or a mixture thereof. In one embodiment, the physiologically acceptable buffer comprises a buffering agent (for example, sodium acetate) at a concentration from about 5 mM to about 30 mM, about 10 mM to about 30 mM, about 15 mM to about 30 mM, about 20 mM to about 30 mM, or about 25 mM to about 30 mM (for example, about 5 mM, about 8 mM, about 10 mM, about 12 mM, about 15 mM, about 18 mM, about 20 mM, about 22 mM, about 25 mM, about 28 mM, or about 30 mM). In some embodiments, the physiologically acceptable buffer comprises the buffering agent (for example, sodium acetate) at a concentration of at least 10 mM.

In some embodiments, the physiologically acceptable buffer comprises an acetate buffer. In some embodiments, the buffering agent is sodium acetate. In some embodiments, the buffering agent is potassium acetate. In some embodiments, the physiologically acceptable buffer comprises an acetate buffer (for example, sodium acetate or potassium acetate) at a concentration of about 5 mM to about 30 mM, for example, about 10 mM to about 20 mM. In some embodiments, the physiologically acceptable buffer comprises an acetate buffer (for example, sodium acetate or potassium acetate) at a concentration of about 10 mM.

In some embodiments, the physiologically acceptable buffer comprises a citrate buffer. In some embodiments, the buffering agent is trisodium citrate. In some embodiments, the buffering agent is magnesium citrate. In some embodiments, the buffering agent is potassium citrate. In one embodiment, the physiologically acceptable buffer comprises the citrate buffer (for example, sodium citrate, magnesium citrate, or potassium citrate) at a concentration from about 5 mM to about 30 mM, for example, about 10 mM to about 20 mM. In some embodiments, the physiologically acceptable buffer comprises the citrate buffer (for example, sodium citrate, magnesium citrate, or potassium citrate) at a concentration of about 10 mM.

In one embodiment, the physiologically acceptable buffer comprises a phosphate buffer. In one embodiment, the physiologically acceptable buffer comprises potassium phosphate. In one embodiment, the physiologically acceptable buffer comprises potassium phosphate at a concentration from about 5 mM to about 30 mM, for example, about 10 mM to about 20 mM. In some embodiments, the physiologically acceptable buffer comprises the phosphate buffer (for example, potassium phosphate) at a concentration of about 10 mM.

In some embodiments, the buffered formulation comprises a tonicity modifier. In some embodiments, the tonicity modifier is mannitol, dextrose, glycerin, lactose, sucrose, trehalose, or a mixture thereof. In some embodiments, the tonicity modifier is mannitol. The use of tonicity modifiers is well known in the medicinal arts, and one of skill in the art can use one or more of the tonicity modifiers disclosed herein to provide a liquid pharmaceutical formulation suitable for subcutaneously or intravenous administration. See, for example, Pramanick et al., Pharma Times., Vol 45, No. 3, (2013); see also, Formulating Poorly Water Soluble Drugs, Williams, Watts, and Miller, eds., Springer Science and Business Media (2011).

In some embodiments, the tonicity modifier or combination of tonicity modifiers is present in the formulation at a concentration of about 20-75 mg/ml, about 20-60 mg/ml, about 25-55 mg/ml, about 30-75 mg/ml, about 30-50 mg/ml, about 35-45 mg/ml, about 40-45 mg/ml, about 45-75 mg/ml, or about 45-60 mg/ml (for example, about 20 mg/ml, about 22 mg/ml, about 25 mg/ml, about 28 mg/ml, about 30 mg/ml, about 32 mg/ml, about 35 mg/ml, about 38 mg/ml, about 40 mg/ml, about 42 mg/ml, about 45 mg/ml, about 48 mg/ml, about 50 mg/ml, about 52 mg/ml, about 55 mg/ml, about 58 mg/ml, about 60 mg/ml, about 65 mg/ml, about 70 mg/ml, or about 75 mg/ml). In some embodiments, the formulation comprises the tonicity modifier at a concentration range of about 30 mg/ml to about 60 mg/ml.

In some embodiments, the tonicity modifier comprises mannitol. In some embodiments, the mannitol is present at a concentration of about 40-50 mg/ml. In some embodiments, the mannitol is present at a concentration in the range of about 40 mg/ml to about 45 mg/ml. In some embodiments, the mannitol is present at a concentration of about 45 mg/ml. In some embodiments, the mannitol is present at a concentration of at least 45 mg/ml.

In one embodiment, the tonicity modifier comprises dextrose. In one embodiment, the dextrose is present at a concentration of about 20 mg/ml to about 60 mg/ml (for example, about 20 mg/ml, about 40 mg/ml, about 45 mg/ml, or about 60 mg/ml).

In one embodiment, the tonicity modifier comprises glycerin. In some embodiments, the glycerin is present at a concentration of about 20 mg/ml to about 60 mg/ml (for example, about 20 mg/ml, about 40 mg/ml, about 45 mg/ml, or about 60 mg/ml).

In one embodiment, the tonicity modifier comprises lactose. In one aspect, the lactose is present at a concentration of about 20 mg/ml to about 60 mg/ml (for example, about 20 mg/ml, about 40 mg/ml, about 45 mg/ml, or about 60 mg/ml).

In one embodiment, the tonicity modifier comprises sucrose. In some embodiments, the sucrose is present at a concentration of about 20 mg/ml to about 60 mg/ml (for example, about 20 mg/ml, about 40 mg/ml, about 45 mg/ml, or about 60 mg/ml).

In one embodiment, the tonicity modifier comprises trehalose. In one aspect, the trehalose is present at a concentration of about 20 mg/ml to about 60 mg/ml (for example, about 20 mg/ml, about 40 mg/ml, about 45 mg/ml, or about 60 mg/ml).

In some embodiments, the buffered formulation comprises two or more tonicity modifiers. In some embodiments, the buffered formulation comprises two or more tonicity modifiers selected from the group consisting of mannitol, dextrose, glycerin, lactose, sucrose, and trehalose. In some embodiments, the buffered formulation comprises mannitol at least one other tonicity modifier.

In some embodiments, the formulation further comprises one or more additional excipients such as preservatives, surfactants (for example, a polysorbate or a poloxamer), or colorants (for example, pharmaceutically acceptable dyes, inorganic pigments, and natural colorants). A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc., each of which is incorporated by reference herein.

In some embodiments, the avexitide formulation is provided as a single-use prefilled syringe, for example, in a kit comprising multiple single-use prefilled syringes (for example, 10, 20, 30, 40, 50, or 60 prefilled syringes). In some embodiments, the single-use prefilled syringe comprises an avexitide formulation comprising avexitide at a concentration of at least 30 mg/mL, a tonicity modifier, and a buffer having a pH in the range of 5.0 to 6.0. In some embodiments, the single-use prefilled syringe comprises avexitide in an amount of at least 20 mg (for example, 20-90 mg, 30-75 mg, 30-60 mg, 40-90 mg, or 60-90 mg, for example, 20 mg, 25 mg, 30 mg, 40 mg, 45 mg, 50 mg, 60 mg, 70 mg, 80 mg, or 90 mg). In some embodiments, the single-use prefilled syringe comprises avexitide in an amount of at least 30 mg. In some embodiments, the avexitide formulation optionally further comprises a preservative.

In some embodiments, the avexitide formulation is provided in a pen injector device. In some embodiments, the pen injector device is a glass device (for example, a glass cartridge pen injector device). In some embodiments, the pen injector device is a single-use device. In some embodiments, the pen injector device comprises an avexitide formulation comprising avexitide at a concentration of at least 30 mg/mL, a tonicity modifier, and a buffer having a pH in the range of 5.0 to 6.0. In some embodiments, the pen injector device comprises avexitide in an amount of at least 20 mg (for example, 20-90 mg, 30-75 mg, 30-60 mg, 40-90 mg, or 60-90 mg, for example, 20 mg, 25 mg, 30 mg, 40 mg, 45 mg, 50 mg, 60 mg, 70 mg, 80 mg, or 90 mg). In some embodiments, the pen injector device comprises avexitide in an amount of at least 30 mg. As a non-limiting example, the pen injector device comprises a formulation that comprises about 30-75 mg of avexitide (for example, about 30 mg or about 60 mg of avexitide), a tonicity modifier and a buffer having a pH in the range of about 5.0 to 6.0. In some embodiments, the avexitide formulation optionally further comprises a preservative.

In some embodiments, the avexitide formulation is provided in a unit or multi-dose glass vial or ampule for administration with the use of a syringe, similar to a glucagon emergency kit. In some embodiments, the avexitide formulation is provided as a disposable or reusable pen device. In some embodiments, the disposable or reusable pen device with an avexitide formulation contains multiple doses (for example, 5, 8, 7, 8, 9, 10, or more) doses of avexitide. In some embodiments, the avexitide formulation is provided as an injectable solution in a single-dose tray containing a vial of an avexitide formulation as described herein, a tonicity modifier, and a buffer having a pH in the range of above 5.0 to 6.0 (for example, 5.1-6.0, 5.2-5.8, or 5.5-6.0, including any of 5.1, 5.2, 5.3, 5.4, 5.5, 5.6, 5.7, 5.8, 5.9, or 6.0) and optionally an appropriate volume of an antimicrobial preservative), a vial connector, a syringe, and one or more needles.

In some embodiments, each dose is administered in a total volume ranging from 0.25-2 ml injectate. In some embodiments, each dose is administered in a total volume ranging from about 0.1 ml or even 0.5 ml, with most subjects administering an injection volume ranging from 0.25-1.5 ml, or from 0.5-1 ml, or from 0.7-1 ml, or from 0.1 ml or even 0.5 ml.

### EXAMPLES

The following examples are provided to illustrate, but not to limit, the claimed invention.

### Example 1. Selected Methods

### A. Buffet Test

A free-choice standard buffet is used to measure total caloric intake and macronutrient distribution in the chosen foods from standard foods of known nutrient composition. Participants choose two of the entrees from a provided list of meals, and in addition they are provided with almond flour crackers, (for example, produced by Fat Snax, Brooklyn, New York), guacamole (for example, Wholly® guacamole by MegaMex foods, Orange, California), and string cheese (for example, low moisture, part skim Lucerne® mozzarella cheese, Guggisberg Cheese, Millersburg, Ohio). The amount of items eaten from the buffet (including partially consumed buffet items) is recorded over the subsequent hour. The total kilocalories of food consumed and macronutrients ingested are analyzed by ESHA (Salem, Oregon) Food Processor® Nutrition Analysis software.

### B. Meal Tolerance Test (MTT)

Subjects undergo a MTT to establish a baseline metabolic profile if not previously screened. The MTT may conducted in the Stanford University Clinical and Translational Research Unit (CTRU) or an equivalent facility. An indwelling catheter is placed into an antecubital vein for collection of blood. Blood samples are collected by the trained staff over the duration of the MTT. At time 0, subjects are given a standardized meal (for example, Ensure® Compact Drink (Abbott Laboratories, Abbott Park, Illinois)), which they must consume within 15 minutes. Blood glucose, insulin, c-peptide, glucagon, GIP, GLP-1, and other incretin hormone levels are measured for multiple timepoints over 3 hours. Symptoms of hypoglycemia are surveyed during the administration of the test. Average total blood drawn per MTT is 100 ml

### C. CGM Placement, Instruction, and Data Capture and Adjustment for Loss of Signal and Dietary Liberalization

Subjects undergo continuous glucose monitoring (CGM) during the outpatient setting. Wearing the CGM devices during the study allows for comparing the accuracy of the CGM meters to detect vast and rapid changes in glycemic values during metabolic provocation. Enrolled participants are scheduled for a video training meeting with the study coordinator. During the first training session, the coordinator explains the study design, goal, and potential risks associated with the CGM device. Participants put on their CGM device(s) under the coordinator supervision and are instructed on how to check their glucose values in real time using a glucometer, as the CGM will be blinded. After completion of the 7-day wear period, participants have another video or in-person meeting with the coordinator to remove the CGM transmitter(s) and return them back to the study team for data capture. Once the CGM transmitters are received by the study team, the data is deidentified and uploaded to Dexcom Clarity, a diabetes management application, through a Dexcom receiver, a component of Dexcom Mobile G6 Pro CGM system (Dexcom, Fort Lauderdale, Florida). The raw data is exported to appropriate software for data analysis.

CGM endpoints include the time spent below 54 mg/dL and 70 mg/dL adjusted for minutes of use and time spent below 54 mg/dL and 70 mg/dL adjusted for glucose spikes greater than or equal to 150 mg/dL indicating consumption of carbs. The data is also adjusted for time of capture. Dietary liberalization is estimated by the number of spikes. Glucose spikes are defined as glucose rising above 150 mg/dL and persisting for at least 15 minutes, with the assumption that this indicates consuming carbohydrate-containing food/drink.

### D. Cronometer Application (Cronometer Software Inc., Revelstoke, British Columbia, Canada)

Food and activity is tracked using a smartphone application Cronometer Pro, to quantify food consumption and nutritional data (macro and micronutrient composition, fiber, etc.), energy balance and activity (minutes, level).

### Example 2. Proof of Concept Seven-Day Study: Evaluation of Avexitide for Improved Eating Behavior in Cancer Patients with Total Gastrectomy or Esophagectomy

### A. Objectives:

The primary efficacy objective is the following:
Evaluate the effect of avexitide on eating behaviors during a standardized buffet meal in the clinic setting, measured as total caloric intake and macronutrient distribution by ESHA (Salem, Oregon) Food Processor® Nutrition Analysis software, feeding interest by Visual Analog Scale (VAS), a psychometric response scale that can be used in questionnaires (see, for example, and appetite and hunger by Council on Nutrition and Appetite Questionnaire (CNAQ); and during ad-lib conditions in the outpatient setting, measured by Cronometer Pro nutrition tracking application (Cronometer Software Inc., Revelstoke, British Columbia, Canada).

The primary safety objective is the following:
Evaluate the safety and tolerability of avexitide.

The secondary efficacy objectives are to evaluate the effect of avexitide on the following:
Gastrointestinal symptoms, as measured by the gastrointestinal short health scale (SHS-GI) and the Gastrointestinal Symptom Rating Scale (GSRS);
Hypoglycemia, fear of hypoglycemia, and associated symptoms, as measured by plasma glucose nadir and the Edinburgh Hypoglycemia Symptom Scale (EHSS) during standardized mixed meal tolerance testing (MMTT) in the clinic setting, by the Hypoglycemia Fear Survey-II (HFS-II), and by continuous glucose monitoring (CGM) and self-monitoring of blood glucose (SMBG), and electronic diary (eDiary) in the outpatient setting.
Evaluate the effect of avexitide on Quality of life (QOL), as measured by and the 36-Item Short-Form Health Survey (SF-36)

### B. Population:

### (i) Inclusion Criteria

Patients who meet all of the following criteria are eligible to participate in the study:
1. Ability to understand the purpose and risks of the study; to be willing and able to adhere to scheduled visits, treatment plans, laboratory tests, procedures related to the use of the smartphone app, and other study evaluations and procedures, and provide written informed consent;
2. At least 18 years of age;
3. Documented total gastrectomy or esophagectomy within 12 months of enrollment; and
4. At least one of the following: undesirable weight loss, expected undesirable weight loss due to the patient being underweight or of normal weight prior to the surgery (e.g., low BMI prior to surgery (BMI<23 kg/m²)), or physician's concern for current or potential malnourishment.

### (ii) Exclusion Criteria:

Patients meeting any of the following criteria are excluded from the study:
1. Participation in another interventional clinical study within 30 days before screening;
2. Donation or loss of >500 mL of blood or blood product within 56 days before the first dose of study drug;
3. Clinically significant active infection within 14 days before the first dose of study drug;
4. History of any clinically relevant acute or chronic psychiatric, renal, hepatic, pancreatic, cardiovascular, neurological, hematological, or gastrointestinal abnormality (e.g., inflammatory bowel disease) or prior malignancy, with the exception of those malignancies for which an upper GI surgery was performed within 12 months before randomization or with the exception of adequately treated basal cell or squamous cell skin cancer, or in situ cervical cancer;
5. Abnormal liver function, defined as transaminases (alanine transaminase (ALT), aspartate transaminase (AST)) levels >2× upper limit of normal (ULN) and/or bilirubin level >2× ULN at screening; abnormal renal function, defined as a glomerular filtration rate (GFR)<60 mL/min/1.73 m² (calculated using the Chronic Kidney Disease Epidemiology Collaboration (CKD-EPI) equation) at screening; abnormal electrolyte concentrations including potassium, magnesium, phosphorus and sodium.

### C. Study Design:

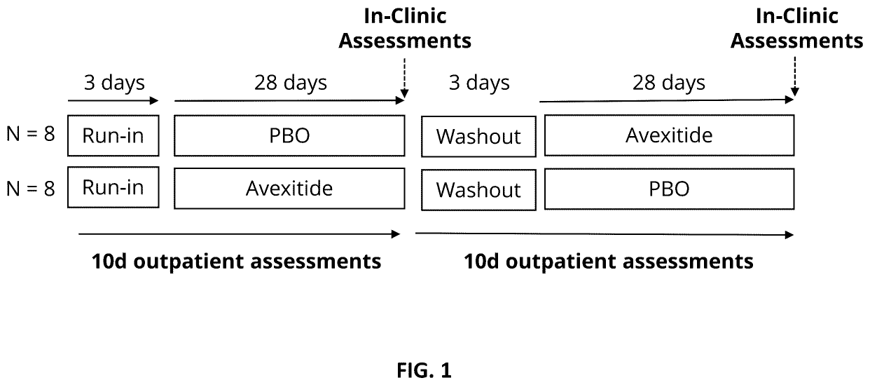

This randomized, placebo-controlled, crossover study is designed to evaluate the effects of avexitide treatment on feeding behaviors, gastrointestinal symptoms, hypoglycemia and quality of life among patients who have previously undergone total gastrectomy or esophagectomy for gastric or esophageal cancer treatment or prophylaxis. This study includes a 3-day CGM run-in period and two 7-day treatment periods separated by a 3-day washout period, as shown in FIG. 1. Approximately 16 participants are randomized 1:1 to 1 of 2 arms, each differing in the order of dosing regimen.

Upon randomization, baseline assessments are completed. Anthropometrics, including height, weight, BMI and body circumference measurements are taken. A standardized buffet meal test is given to quantify energy intake and composition by ESHA Food Processor Feeding interest, appetite, hunger, satiety, and taste aversion will be assessed by VAS and CNAQ. A standardized MMTT is performed to assess glucose and hormone profiles and hypoglycemia and gastrointestinal symptoms during meal provocation with symptoms of hypoglycemia assessed by EHSS. Additionally, patients complete the GSRS and SHS-GI questionnaires to assess GI symptoms and the HFS-II and SF-36 to assess fear of hypoglycemia and health-related quality of life.

Starting at the beginning of the CGM run-in period and throughout Treatment Periods 1 and 2 (inclusive of the 3-day washout period), avexitide or placebo is administered in the amount of 45 mg twice daily in crossover design and random order, for a total of 10 days of avexitide or placebo treatment. Throughout each 7-day treatment period in the home setting, participants document ad-lib energy intake via Cronometer Pro application food logging and document hypoglycemic events via an electronic diary (eDiary; Medpace, Inc., Cincinnati, Ohio), self-monitoring of blood glucose (SMBG), and blinded continuous glucose monitoring (CGM). At the end of each treatment period, participants return to the site to repeat baseline assessments. A 3-day washout period occurs in between treatment periods 1 and 2.

### D. Study Procedures:

(i) in-Clinic Assessments

Buffet Test: At baseline and at the end of each treatment period, subjects are admitted to the clinic after an overnight fast to undertake a free-choice standard buffet test to measure total caloric intake and macronutrient distribution in the chosen foods from standard foods of known nutrient composition (e.g., vegetable lasagna (Stouffers®, Nestlé USA, Inc., Solon, OH); vanilla pudding (Hunts, KraftHeinz North America, Tarrytown, NY); and skim milk). The amount of items eaten from the buffet (including partially consumed buffet items) will be recorded over the subsequent hour. The total kilocalories of food consumed and macronutrients ingested will be analyzed by validated software (ProNutra™ 3.0; Viocare® Technologies, Inc., Princeton, NJ). Additionally, the VAS and CNAQ will be used to quantify feeding interest, appetite, hunger and taste aversion.

Mixed meal tolerance test (MMTT): At baseline and at the end of each treatment period, subjects are admitted to the clinic after an overnight fast for 180-minute MMTT. After a baseline blood draw, subjects consume 2 Ensure® Compact Drinks (Abbott Laboratories, Abbott Park, Illinois) containing 64 g of carbohydrate over 10 minutes, with labs drawn every 15 minutes (for plasma glucose, insulin, c-peptide, GLP-1, and glucagon) and bedside assessment of point of care glucose measurements collected every 30 minutes. If rescue parameters are met (the earlier of point of care glucose ≤50 mg/dL with documented neuroglycopenic symptoms or ≤40 mg/dL irrespective of symptoms), final blood samples are drawn and participants rescued by intravenous dextrose. Plasma glucose and hormones (glucose, insulin, glucagon, GLP-1, GIP) are collected every 30 minutes and assayed per standard methods.

To assess the temporal presence and severity of autonomic, neuroglycopenic and non-specific symptoms, a five-point Likert severity gradation (0=none; 5=severe) is imposed on the EHSS—a survey validated for use in insulin-treated diabetic patients for identification of symptoms of acute hypoglycaemia. The severity-graded EHSS is completed by participants every 30 min prior to each blood draw during each oral glucose tolerance test (OGTT). Symptoms assessed include autonomic (sweating, shaking, palpitations, hunger), neuroglycopenic (blurred vision, confusion, drowsiness, odd behaviour, speech difficulty, incoordination, dizziness, inability to concentrate) and malaise (nausea, headache). For each participant, a composite score for all timepoints is recorded. To isolate symptoms associated temporally with the glucose rise period (from T=0 to the individual participant's glucose peak) and the glucose fall period (from glucose peak to nadir), two sub-scores are included: “glucose rise” and “glucose fall” scores. Analysis further group symptom scores according to symptom type: autonomic, neuroglycopenic, or malaise.

Questionnaires: At baseline and at the end of each treatment period, subjects complete the SHS-GI, GSRS, HSF-II and SF-36. Questionnaires must be completed without any external assistance using a ballpoint pen with black ink prior to any study procedures or lab draws, in a quiet and private setting, and in an unrushed manner. Each questionnaire is reviewed to ensure all questions have been answered. If any questions do not have a response, the study participant is asked if they would like to complete a response. If the study participant does not wish to respond to a question, no further action is taken, and the completed questionnaire(s) are filed in the source document folder.

### (ii) Outpatient Assessments:

CGM, SMBG, eDiary use in the outpatient setting: Throughout the two 7-day treatment periods, subjects use an eDiary (internet-connected web application), a CONTOUR® Next One glucometer (Ascensia Diabetes Care, Parsippany, New Jersey), and blinded Dexcom Mobile G6 Pro CGM system (Dexcom, Fort Lauderdale, Florida) for recording of hypoglycemic events occurring in the ambulatory setting. For each episode, patients record hypoglycemia symptoms/signs, the lowest SMBG reading during the episode, actions taken to treat or prevent the episode, requirement for assistance, and whether the episode was postprandial. Study drug injections are also recorded, and adherence is monitored via accounting of returned study drug vials.

Cronometer Pro app: Throughout the two 7-day treatment periods, food and activity is tracked using the smartphone Cronometer Pro app, along with Nutrition Tracking Software for Professionals, to quantify food consumption and nutritional data (macro and micronutrient composition, fiber, etc.), energy balance and activity (minutes, level).

### E. Endpoints:

The primary efficacy endpoint is total caloric intake (kcal) during the standardized buffet test.

Secondary endpoints supporting the primary objective include the following:
Feeding interest, measured by VAS before the buffet test.
Appetite and hunger, measured by CNAQ.
Taste aversion, measured by an exploratory tool.
Total energy intake, measured by Chronometer application over 7 days by food logging.

Secondary endpoints supporting secondary objectives include the following:
Gastrointestinal symptoms, measured by SHS-GI and GSRS at the end of each treatment period.
Magnitude of hypoglycemia, measured as plasma glucose nadir during standardized MMTT.
Autonomic and neuroglycopenic symptoms of hypoglycemia, measured by EHSS score during MMTT provocation.
Hypoglycemic fear-associated worries and behaviors, measured by HFS-II.
Hypoglycemia event rate (<70 mg/dl) measured by SMBG.
Clinically important hypoglycemia event rate (<54 mg/dL) measured by SMBG.
Time in range (80-120 mg/dL) and time below range (<70 mg/dL; <54 mg/dL), measured by blinded Dexcom G6 CGM at home for 7 days during each treatment period.
Quality of life, measured by SF36.

### Example 3. Phase 2 28-Day Study: Evaluation of Avexitide for Improved Nutritional Status and Prevention of Unwanted Weight Loss in Cancer Patients with Total Gastrectomy or Esophagectomy

### A. Objectives:

The primary efficacy objective is: Evaluate the effect of avexitide on body weight, composition, and body mass index (BMI), measured at baseline and at the end of each treatment period by standardized methods for height, weight, body circumference and body impedance analysis (BIA), dual-energy X-ray absorptiometry (DEXA) or computed tomography (CT).

The primary safety objective is: Evaluate the safety and tolerability of avexitide.

The secondary efficacy objectives are to evaluate the effect of avexitide on the following:
Nutritional status, measured by macronutrient composition of meals, blood albumin levels and/or other biomarkers;
Physical performance, measured by via total activity and/or grip force;
Eating behaviors and energy balance measured during a standardized buffet meal in the clinic setting as total caloric intake and macronutrient distribution by ESHER food processor, feeding interest by Visual Analog Scale (VAS), and appetite, taste, satiety and hunger by Council on Nutrition and Appetite Questionnaire (CNAQ);
Energy balance, dietary composition, and dietary liberalization during ad-lib conditions in the outpatient setting, measured by Cronometer Pro application, CGM, and eDiary;
Gastrointestinal symptoms, as measured by the gastrointestinal short health scale (SHS-GI) and the Gastrointestinal Symptom Rating Scale (GSRS);
Hypoglycemia, fear of hypoglycemia, and associated symptoms, as measured by plasma glucose nadir and the Edinburgh Hypoglycemia Symptom Scale (EHSS) during standardized mixed meal tolerance testing (MMTT) in the clinic setting, by the Hypoglycemia Fear Survey-II (HFS-II), and by continuous glucose monitoring (CGM) and self-monitoring of blood glucose (SMBG), and electronic diary (eDiary) in the outpatient setting.

Quality of life (QoL), as measured by and the 36-Item Short-Form Health Survey (SF-36).

### B. Population:

### (i) Inclusion Criteria

Patients who meet all of the following criteria will be eligible to participate in the study:
1. Ability to understand the purpose and risks of the study; to be willing and able to adhere to scheduled visits, treatment plans, laboratory tests, procedures related to the use of the smartphone application, and other study evaluations and procedures, and provide written informed consent;
2. At least 18 years of age;
3. Documented total gastrectomy or esophagectomy within 12 months of enrollment; and
4. At least one of the following: undesirable weight loss, expected undesirable weight loss due to the patient being underweight or of normal weight prior to the surgery (e.g., low BMI prior to surgery (BMI<23 kg/m²)), or physician's concern for current or potential malnourishment.

### (ii) Exclusion Criteria:

Patients meeting any of the following criteria are excluded from the study:
1. Participation in another interventional clinical study within 30 days before screening;
2. Donation or loss of >500 mL of blood or blood product within 56 days before the first dose of study drug;
3. Clinically significant active infection within 14 days before the first dose of study drug;
4. History of any clinically relevant acute or chronic psychiatric, renal, hepatic, pancreatic, cardiovascular, neurological, hematological, or gastrointestinal abnormality (e.g., inflammatory bowel disease) or prior malignancy, with the exception of those malignancies for which an upper GI surgery was performed within 12 months before randomization or with the exception of adequately treated basal cell or squamous cell skin cancer, or in situ cervical cancer;
5. Abnormal liver function, defined as transaminases (alanine transaminase (ALT), aspartate transaminase (AST)) levels >2× upper limit of normal (ULN) and/or bilirubin level >2× ULN at screening; abnormal renal function, defined as a glomerular filtration rate (GFR)<60 mL/min/1.73 m² (calculated using the Chronic Kidney Disease Epidemiology Collaboration (CKD-EPI) equation) at screening; abnormal electrolyte concentrations including potassium, magnesium, phosphorus and sodium.

### C. Study Design:

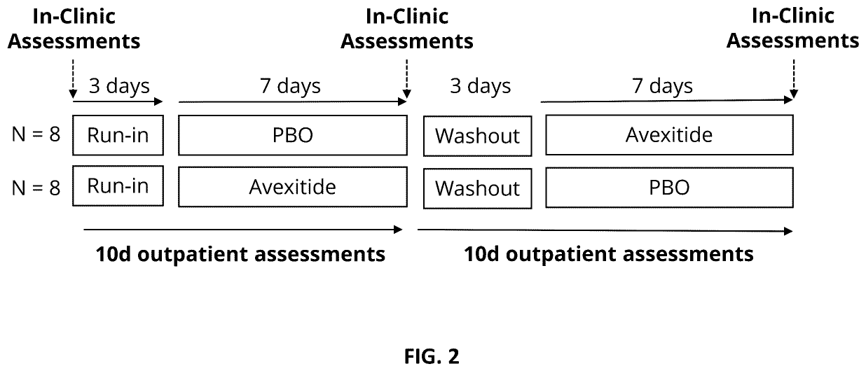

This is a Phase 2, single- or multi-center, placebo-controlled, cross-over study of avexitide injection in approximately 16 patients with gastrointestinal surgery. Eligible participants must have undergone total gastrectomy or esophagectomy within 12 months of enrollment. Study subjects also have at least one of undesirable weight loss, expected undesirable weight loss due to the patient being underweight or of normal weight prior to the surgery (e.g., low BMI prior to surgery (BMI≤23 kg/m²)), or physician's concern for current or potential malnourishment. Enrolled participants are randomized to one of two 28-day avexitide or placebo dosing regimen orders (FIG. 2). Doses are self-administered in the outpatient setting via subcutaneous (SC) injection. Throughout the study duration participants are instructed to maintain post-GI surgery dietary guidelines.

Prior to Treatment Period 1 (at baseline) and at the end of Treatment Periods 1 and 2, participants undergo various in-clinic assessments, including body height/weight/BMI, body composition via BIA, DEXA, or CT, have fasting and postprandial laboratory assessments to measure blood glucose, insulin and incretin hormones (e.g., GLP-1), as well as measures to assess nutritional status (e.g., albumin), physical performance assessment (e.g., via total activity and/or grip force), a standardized buffet test, and patient questionnaires (e.g., to assess gastrointestinal symptoms; appetite, taste and satiety; fear of hypoglycemia-related behaviors). Throughout Treatment Periods 1 and 2, patients track feeding behaviors and energy balance through use of the Chronometer application in addition to an electronic diary (eDiary). Additionally, hypoglycemia events will be tracked by blinded continuous glucose monitor (CGM), self-monitoring of blood glucose (SMBG), and electronic diary (eDiary) entries throughout study conduct. At the end of Treatment Period 2, participants return used and unused study drug vials during the safety follow-up visit.

### D. Study Procedures:

(i) in-Clinic Assessments

Anthropometrics: Height, weight, body mass index (BMI), and body circumference (waist, hip, and limbs) are measured after an overnight fast at baseline, with weight, BMI and body circumference repeated after overnight fast at the end of treatment periods 1 and 2. Body composition: Body composition is measured (lean body mass, fat mass and free fat mass, and water) at baseline and at the end of treatment periods 1 and 2 and analyzed by body impedance analysis (BIA), dualenergy X-ray absorptiometry (DEXA) or (computed tomography (CT), following standard methods.

Buffet Test: At baseline and at the end of each treatment period, subjects are admitted to the clinic after an overnight fast to undertake a free-choice standard buffet test to measure total caloric intake and macronutrient distribution in the chosen foods from standard foods of known nutrient compositions, e.g., vegetable lasagna (Stouffers, Nestle USA, Inc., Solon, OH)m vanilla pudding (Hunts, KraftHeinz North America, Tarrytown, NY), and skim milk. The amount of items eaten from the buffet (including partially consumed buffet items) is recorded over the subsequent hour. The total kilocalories of food consumed and macronutrients ingested will be analyzed by validated software (ProNutra 3.0; Viocare Technologies, Inc., Princeton, NJ). Additionally, the VAS and CNAQ are used to quantify feeding interest, appetite, hunger and taste aversion.

Mixed meal tolerance test (MMTT): At baseline and at the end of each treatment period subjects are admitted to the clinic after an overnight fast for 180-minute MMTT. After a baseline blood draw, subjects consume 2 Ensure Compact Drinks containing 64 g of carbohydrate over 10 minutes, with labs drawn every 15 minutes (for plasma glucose, insulin, c-peptide, GLP-1, and glucagon)m and bedside assessment of point of care glucose measurements is collected every 30 minutes. If rescue parameters are met (the earlier of point of care glucose≤50 mg/dL with documented neuroglycopenic symptoms or ≤40 mg/dL irrespective of symptoms), final blood samples are drawn and participants are rescued by intravenous dextrose. Plasma glucose and hormones (glucose, insulin, glucagon, GLP-1, GIP) are collected every 30 minutes and assayed per standard methods.

To assess the temporal presence and severity of autonomic, neuroglycopenic and non-specific symptoms, a five-point Likert severity gradation (0=none; 5=severe) is imposed on the EHSS—a survey validated for use in insulin-treated diabetic patients for identification of symptoms of acute hypoglycemia. The severity-graded EHSS is completed by participants every 30 min prior to each blood draw during each OGTT. Symptoms assessed include autonomic (sweating, shaking, palpitations, hunger), neuroglycopenic (blurred vision, confusion, drowsiness, odd behavior, speech difficulty, incoordination, dizziness, inability to concentrate) and malaise (nausea, headache). For each participant, a composite score for all timepoints is recorded. To isolate symptoms associated temporally with the glucose rise period (from T=0 to the individual participants' glucose peak) and the glucose fall period (from glucose peak to nadir), two sub-scores are included, “glucose rise” and “glucose fall” scores. Analysis further groups symptom scores according to symptom type: autonomic, neuroglycopenic or malaise. Questionnaires: At baseline and at the end of each treatment period, subjects complete the SHS-GI, GSRS, HSF-II and SF-36

### (ii) Outpatient Assessments:

CGM, SMBG, eDiary use in the outpatient setting: Throughout the two treatment periods, subjects use an eDiary (internet-connected web application), a CONTOUR® Next One glucometer, and blinded Dexcom Mobile G6 Pro CGM for recording of hypoglycemic events occurring in the ambulatory setting. For each episode, patients record hypoglycemia symptoms/signs, the lowest SMBG reading during the episode, actions taken to treat or prevent the episode, requirement for assistance, and whether the episode was postprandial. Study drug injections are be recorded, and adherence is monitored via accounting of returned study drug vials.

Cronometer Pro application: Throughout the two treatment periods, food and activity is tracked using the smartphone Cronometer Pro application, along with Nutrition Tracking Software for Professionals, to quantify food consumption and nutritional data (macro and micronutrient composition, fiber, etc.), energy balance and activity (minutes, level).

### E. Endpoints:

The primary endpoint is percent change in body weight from baseline, measured at the end of each treatment period

Secondary endpoints supporting the primary objective include:
Percent change BMI from baseline, measured at the end of each treatment period; and
Percent change in body composition (lean body mass, fat mass and free fat mass, and water) from baseline, as analyzed by body impedance analysis (BIA), dualenergy X-ray absorptiometry (DEXA) or (computed tomography (CT).

Secondary endpoints supporting secondary objectives include percent change from baseline in:
Physical performance, as measured, for example, by total activity and grip strength;
GI symptoms, as measured, for example, by CNAQ questionnaire;
Appetite, perception of taste, and perception of satiety, as measured, for example, by SHS-GI questionnaire;
Hypoglycemic fear-associated worries and behaviors, as measured by HSF-II questionnaire;
Feeding behavior and liberalization of carbohydrate intake, as assessed in-clinic during a standardized buffet test and on an ongoing basis in the outpatient setting using food and activity tracking using a suitable smartphone application, such as Cronometer Pro, Nutrition Tracking Software for Professionals, to quantify food consumption and nutritional data (macro and micronutrient composition, fiber, etc.), energy balance and activity (minutes, level);
Nutritional status as measured, for example, by albumin or other biomarkers;
Fasting and postprandial glucose and hormone profiles (e.g., peak, nadir, AUC insulin, glucagon, GLP-1, GIP levels) during MMTT testing;
Rate of levels 1-3 hypoglycemia as measured by CGM and SMBG/eDiary.

### Example 4. Study of Avexitide Therapy in Patients with Severe Hypoglycemia after Gastrointestinal Surgery, Including RYGB, VSG, Esophagectomy, Gastrectomy, and Nissen Fundoplication

### A. Study Description:

Phase 2b, single center, open-label, cross-over study of avexitide injection was conducted in 16 patients with surgically-induced severe, diet-refractory hyperinsulinemic hypoglycemia (HH). Eligible participants had a confirmed diagnosis of HH after upper GI surgery and demonstrated severe, diet refractory HH, as documented by presence of at least two severe hypoglycemia events from which at least one must be characterized by glucose <54 mg/dL as measured by CGM during the 14-day Run-in Period while adhering to medical nutritional therapy.

Eligible participants included patients with HH occurring after bariatric surgery (Roux-en-Y Gastric Bypass (RYGB) or Vertical Sleeve Gastrectomy (VSG)) or other upper-gastrointestinal surgery (gastrectomy, esophagectomy, or Nissen Fundoplication).

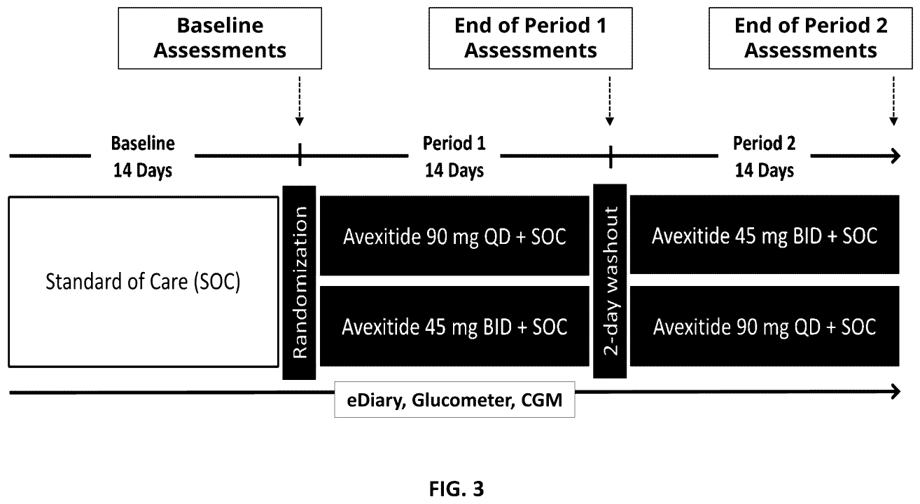

Subjects meeting initial eligibility requirements underwent a 14-day Baseline Run-In consisting of standard of care (SOC) treatment (medical nutrition therapy) with standardized mixed meal consumption and screening labs performed at the end of the 14-day SOC Baseline Period to confirm eligibility and establish a baseline within 90 days of randomization. Enrolled participants completed were randomized to one of two 14-day avexitide dosing regimen orders (FIG. 3). Doses were self-administered in the outpatient setting via subcutaneous (SC) injection. Participants were instructed to maintain standard of care medical nutrition therapy throughout all three treatment periods (Baseline, Treatment Period 1 and Treatment Period 2) following standard dietary guidelines for the management of hypoglycemia (frequent small, low glycemic index meals and snacks containing up to 30 and 15 g of carbohydrates (CHO), respectively, with elimination of all simple sugars). Hypoglycemia assessed throughout by blinded CGM, patient electronic diary (eDiary) and SMBG. At the end of each of the Baseline Period, Treatment Period 1, and Treatment Period 2, participants completed quality of life (QoL) assessments (SF-36, EQ-5D) and had labs drawn, including trough PK samples. Used and unused study drug vials were returned with accountability conducted during the safety follow-up visit, which occurred at the end of Treatment Period 2.

For the subset of patients who had undergone gastrectomy or esophagectomy, additional assessments were completed, as this population is known to experience rapid and involuntary weight loss as a result of Post-Gastrectomy Syndrome, characterized by a lowered tolerance for large meals, rapid emptying of food into the small intestine or “dumping,” abdominal cramping pain, diarrhea, lightheadedness after eating as well as increased heart rate and sharp drops in blood sugar levels. Additional assessments conducted in this cohort included anthropometrics, assessment of appetite and hunger, GI symptoms, and QoL assessments were conducted at baseline and at the end of each of Treatment Periods 1 and 2, including body weight, height and BMI, the Council on Nutrition and Appetite Questionnaire (CNAQ), the Short Health Scale-GI (SHS-GI) questionnaire, and the Hypoglycemia Fear Survey-II (HSF-II).

### B. Results:

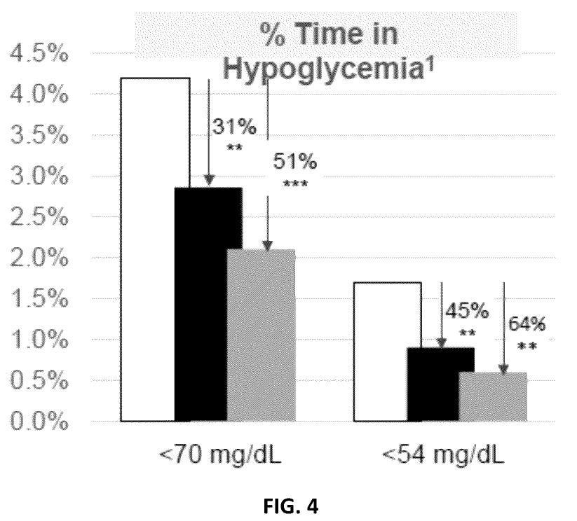

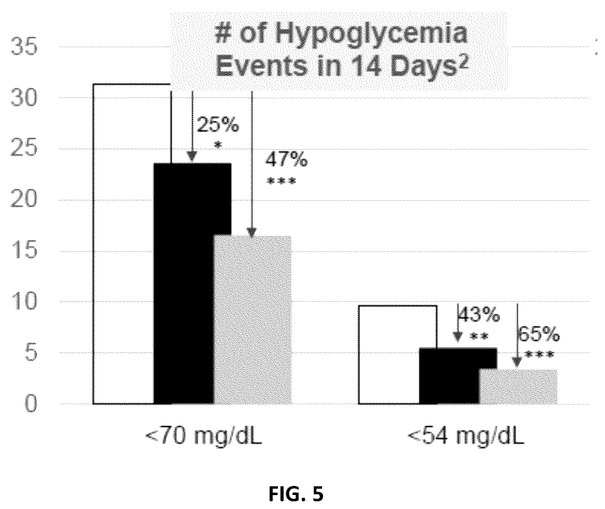

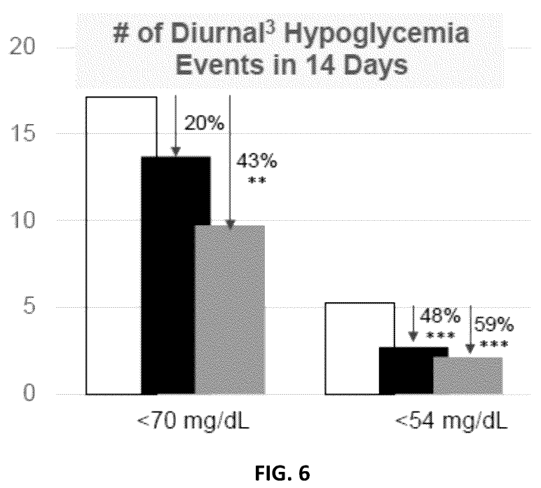

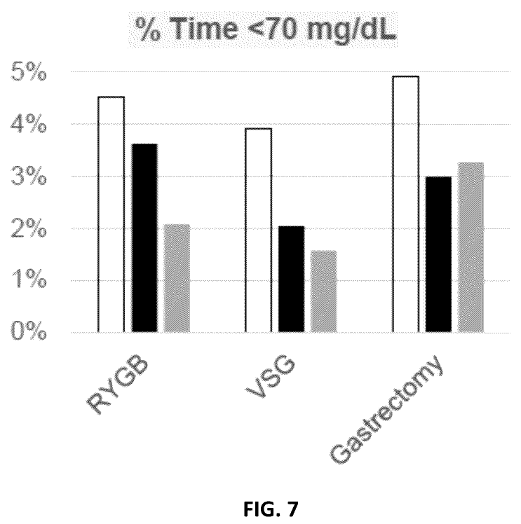

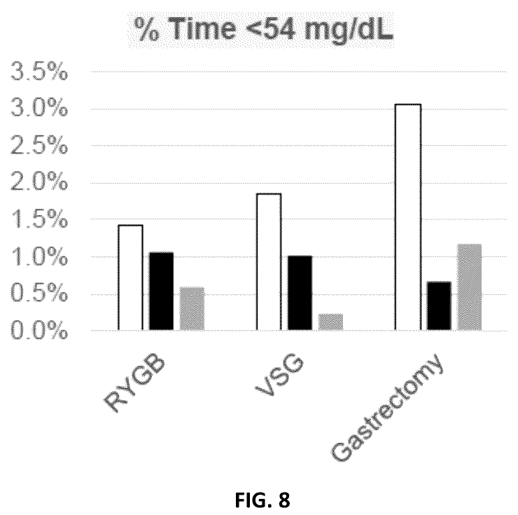

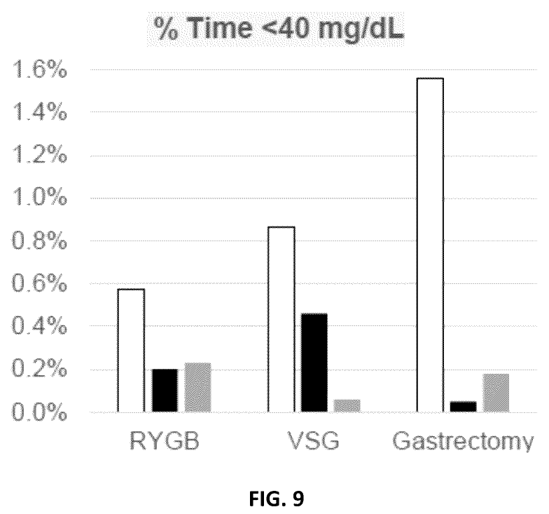

Participant baseline demographic and clinical characteristics are shown in Table 4. Metabolic responses across all study participants as measured by SMBG, eDiary and CGM are shown in Table 5, FIG. 4, FIG. 5, FIG. 6, FIG. 7, FIG. 8, and FIG. 9.

TABLE 4
Participant baseline demographic and clinical characteristics.

| | Treatment Sequence | | |
|---|---|---|---|
| | Baseline, | Baseline, | |
| | avexitide 45 mg | avexitide 90 mg | |
| | twice daily, | once daily, | |
| | avexitide | avexitide | |
| | 90 mg once | 45 mg twice | Total |
| Characteristic | daily (N = 8) | daily (N = 8) | (N = 16) |

Demographics/Anthropometric Characteristics

| | | | | | | |
|---|---|---|---|---|---|---|
| Sex, female/male, n (%) | 6/2 | (75.0/25.0) | 8/0 | (100.0/0) | 14/2 | (87.5/12.5) |
| Age, mean (SD), years | 48.7 | (14.4) | 46.8 | (10.9) | 47.8 | (12.8) |
| Weight, mean (SD), kg | 78.8 | (17.3) | 80.0 | (11.7) | 79.4 | (14.8) |
| BMI, mean (SD), kg/m2 | 28.2 | (6.5) | 28.6 | (3.5) | 28.4 | (5.2) |

Race, n (%)

White
American Indian or Alaskan
Native
Other

Ethnicity, n (%)

Hispanic or Latino
Not Hispanic or Latino

Surgical Subtype

| RYGB, (%) | 4 | (50.0) | 5 | (62.5) | 9 | (56.2) |
|---|---|---|---|---|---|---|
| Nissen, (%) | 1 | (12.5) | 0 | | 1 | (5.2) |
| VSG, (%) | 1 | (12.5) | 3 | (37.5) | 4 | (25.0) |

| | | | | | |
|---|---|---|---|---|---|
| Gastrectomy (%) | 2 | (25.0) | 0 | 2 | (12.5) |

Clinical baseline/history

| | | | | | | |
|---|---|---|---|---|---|---|
| Time since surgery, mean (SD), | 67.6 | (46.1) | 85.1 | (54.1) | 76.3 | (51.0) |
| months | | | | | | |
| Time to first experience of | 11 | (10.9) | 18.2 | (27.4) | 15.4 | (22.1) |
| postprandial hypoglycemia, mean | | | | | | |
| (SD), months | | | | | | |
| History of LOC due to HH, n (%) | 3 | (37.5) | 4 | (50.0) | 7 | (43.7) |

| | | | | | |
|---|---|---|---|---|---|
| History of hospitalization due to | 0 | 1 | (12.5) | 1 | (6.2) |

HH, n (%)

Frequency of symptoms of hypoglycemia

| Daily, n (%) | 6 | (75.0) | 4 | (50.0) | 10 | (62.5) |
|---|---|---|---|---|---|---|
| Weekly, n (%) | 2 | (25.0) | 3 | (37.5) | 4 | (25.0) |
| Very rarely, n (%) | 0 | | 1 | (12.5) | 1 | (6.2) |
| History of type 2 DM before | 0 | | 0 | | 0 | |
| surgery, n (%) | | | | | | |
| History of pharmacotherapy for | 7 | (87.5) | 4 | (50.0) | 11 | (68.7) |

| | | | | | |
|---|---|---|---|---|---|
| HH, n (%) | | | | | |
| History of surgery for HH, n (%) | 1 | (12.5) | 0 | 1 | (6.2) |

TABLE 5
Rate of Level 1-3 hypoglycemia as measured by SMBG and eDiary.

| | Treatment Period (n = 16) | | | | | | |
|---|---|---|---|---|---|---|---|
| | | | Avexitide 45 mg BID | | | Avexitide 45 mg BID | |
| Parameter | | | % | | Mean | % | |
| measured by | Baseline | | Decrease | | event | Decrease | |
| eDiary and | Mean | Mean | from | | rate | from | |
| SMBG | (SD) | (SD) | baseline | p-value | (SD) | baseline | p-value |
| Rate¹ of Level 1 | 5.9 | 2.7 | 53.8% | 0.0028 | 1.9 | 67.5% | 0.0005 |
| Hypoglycemia² | (5.23) | (4.32) | | | (3.78) | | |
| Rate of Level 2 | 2.7 | 1.2 | 56.8% | 0.0027 | 1.3 | 53.3% | 0.0043 |
| Hypoglycemia³ | (3.10) | (1.42) | | | (3.72) | | |
| Rate of Level 3 | 2.5 | 0.8 | 67.5% | 0.0003 | 0.9 | 66.1% | 0.0003 |
| Hypoglycemia⁴ | (3.18) | (1.22) | | | (2.88) | | |

¹Rate is defined as number of episodes in each treatment period normalized to 14 days
²Level 1 Hypoglycemia is defined as SMBG concentrations <70 mg/dL
³Level 2 Hypoglycemia is defined as SMBG concentrations <54 mg/dL
⁴Level 3 Hypoglycemia is defined as a severe event characterized by altered mental and/or physical functioning that requires assistance from another person for recovery. This applies regardless of whether a patent receives external assistance.

Both avexitide treatment regimens resulted in significant reductions in the rates of Levels 1 to 3 hypoglycemia relative to baseline. The rate of SMBG<70 mg/dL (Level 1 hypoglycemia) was reduced by 54% (P=0.003) and 68% (P=0.001) and the rate of SMBG<54 mg/dL (Level 2 hypoglycemia) was reduced by 57% (P=0.003) and 53% (P=0.004) during avexitide 45 mg twice daily and 90 mg once daily, respectively, compared with baseline. The rate of Level 3 events was reduced by 68% (P<0.001) and 66% (P<0.001) during avexitide 45 mg twice daily and 90 mg once daily, respectively.

Blinded CGM corroborated the results observed by SMBG and eDiary. Specifically, the mean percent time with glucose <54 mg/dL was reduced by 45% and 64% during avexitide 45 mg twice daily and 90 mg once daily treatment, respectively. Similarly, the mean number of hypoglycemia events with glucose <54 mg/dL as captured by CGM was reduced by 43% and 65% during avexitide 45 mg twice daily and 90 mg once daily, respectively.

When the patients were evaluated by surgical subtype, it was observed that gastrectomy patients represented a more severe phenotype, demonstrating more percent time at baseline with CGM values <70, <54, and <40 mg/dL than their bariatric (RYGB and VSG) counterparts. A marked amplification of the avexitide treatment effect was also observed among the total gastrectomy cohort as compared to the bariatric patients. Specifically, a greater magnitude of reduction in percent time <54 mg/dL and <40 mg/dL was observed among the total gastrectomy patients. The above results are illustrated in FIG. 7, FIG. 8, and FIG. 9.

A summary of the results from additional assessments (anthropometrics, CNAQ, SGS-GI, HFS-II) conducted in two gastrectomy patients are shown in Table 6.

### (i) Patient 1

Patient 1 was a 23-year-old male with a germline CDH1 gene mutation, who, approximately 5 months prior to study participation, had undergone prophylactic laparoscopic gastrectomy with Roux-en-Y reconstruction and esophago-jejunal anastomosis. Within the first month after surgery he developed postprandial hypoglycemia: fasting glucoses were approximately in the 60-69 mg/dL range. After eating, blood glucose concentrations would spike as high as 200 mg/dL, and then, within 1.5-2 hours after eating, blood glucose concentrations would fall dangerously low (documented as 37 mg/dL by SMBG and CGM). Such episodes occurred multiple times per day, with the lowest values following the highest post-meal spikes. The patient did not have a history of loss of consciousness, seizure, or requirement for emergency medical assistance, but he reported “brain fog” and being “very tired.” Review of CGM data showed multiple glucose values <54 mg/dL in the absence of symptoms. Thus, the patient was determined to have hypoglycemia unawareness. The patient saw a nutritionist and was instructed to avoid carbohydrates and sugars almost completely with no more than 4-7 g of carbohydrates (CHO) per meal. He had lost 14.8% of his body weight (from 155 to 122 lbs.), which was of major concern to the patient, family, and health care team. Severe dietary restrictions were thought to contribute to the patient's weight loss. He also complained of chronic nausea/vomiting, which further limited his oral intake and weight gain.

At baseline, the patient had a body weight of 126 lbs. After 14 days of avexitide administration at 45 mg BID, the patient regained 4.6 lbs. (increase of 3.6% body weight). After additional 14 days of avexitide administration at 90 mg QD, the patient regained another 0.8 lb. (total increase of 4.3% body weight). While the patient had been instructed to adhere to the protocol and maintain a consistent diet throughout all treatment periods, review of blinded CGM data demonstrated dietary liberalization during each 45 mg BID and 90 mg QD administration period as compared to baseline. Dietary liberalization was quantified as number of glucose spikes ≥150 mg/dL, based on the assumption that on average 1 g of CHO raises blood glucose by 3-4 mg/dL, and that fasting glucose was on average 80 mg/dL, such that an increase to 150 mg/dL represented approximately 20 of CHO intake. The number of glucose spikes per day, representing higher CHO meals, increased from 1.44/day at baseline to 3.57/day on avexitide administered at 45 mg BID and 5.33/day on avexitide administered at 90 mg QD, representing a 270% increase in the number of higher carbohydrate meals on avexitide treatment, as compared to baseline.

The patient's CNAQ score at baseline was 23, indicating poor appetite and significant risk of at least 5% weight loss within 6 months and of malnutrition. However, after 14 and 28 days of avexitide treatment, the CNAQ score increased to 24 and 26, respectively, demonstrating reduced risk of weight-loss and malnutrition. GI symptomatology, as measured by SHS-GI, was reduced by over 5% after 28 days of treatment. At baseline, the patient's HFS-II—Worries score was 49. After 14 and 28 days of avexitide treatment, his HFS-II score fell to 42 and 39, respectively, representing a 20.4% reduction in hypoglycemia-associated worries by the end of the trial.

In summary, in this 23-year-old male post-gastrectomy patient with low body weight, poor appetite, gastrointestinal symptoms, and frequent severe hypoglycemia, 28 days of avexitide treatment effectively prevented continued weight loss and, in fact, promoted weight regain. Based on CNAQ, SGS-GI and HFS-II results, the mechanisms mediating weight stabilization were likely multifactorial and complementary, including increased appetite, hunger, and taste sensitivity, reduced gastrointestinal symptoms, and reduced fear of hypoglycemia due to effective prevention of hypoglycemia. In concert, the effects promoted dietary liberalization and improved the patient's weight and nutritional status over the course of a 28-day period.

TABLE 6
Individual Gastrostomy Patient Responses to
Avexitide Treatment by Treatment Period.

| Patient | | | Treatment | Treatment |
|---|---|---|---|---|
| No. | Variable | Baseline | Period 1 | Period 2 |
| 1 | Weight | 126.2 | 130.8 | 131.6 |
| | % Increase | N/A | 3.6% | 4.3% |
| | BMI | 19.2 | 19.9 | 20 |
| | % Increase | N/A | 3.6% | 4.2% |
| | Dietary liberalization | 1.44 | 3.57 | 5.33 |
| | % Increase | N/A | 147.9% | 270.1% |
| | CNAQ | 23 | 24 | 26 |
| | % Increase | N/A | 4.3% | 13.0% |
| | SGS-GI | 290 | 290 | 275 |
| | % Decrease | N/A | 0.0% | 5.2% |
| | HFS-II Worries | 49 | 42 | 39 |
| | % Decrease | N/A | 14.3% | 20.4% |
| 2 | Weight | 125 | 124 | 124 |
| | % Increase | N/A | −0.8% | −0.8% |
| | BMI | 18.5 | 18.3 | 18.3 |
| | % Increase | N/A | −1.1% | −1.1% |
| | Dietary liberalization | 6.57 | 5.87 | 6.57 |
| | % Increase | N/A | −10.7% | 0.0% |
| | CNAQ | 22 | 28 | 26 |
| | % Increase | N/A | 27.3% | 18.2% |
| | SGS-GI | 240 | 80 | 190 |
| | % Decrease | N/A | 66.7% | 20.8% |
| | HFS-II Worries | 29 | 29 | 26 |
| | % Decrease | N/A | 0.0% | 10.3% |

### (ii) Patient 2

Patient 2 was a 72-year-old male with history of gastric cancer, who had undergone total gastrectomy 2.75 years prior to study participation. The patient had been placed on a liquid diet for 6 weeks after surgery and had initially lost 20 lbs. but otherwise fared well in the immediate postoperative period. However, over the course of the following several months, during which he underwent 2 rounds of chemotherapy, he continued to lose weight in an uncontrolled manner, dropping from 174 lbs. to 111 lbs. at his weight nadir. Dietary interventions involving frequent consumption of small, low glycemic index meals failed to mitigate the weight loss. The meals caused pain, gastrointestinal symptoms, and frequent diarrhea (per patient report). The patient also complained of weakness, lethargy and general feeling of despondency. By approximately 1.5 years after surgery, the patient's body weight had stabilized at 125 lbs., however, he continued to experience low appetite, taste and feeding aversion, low energy, and gastrointestinal symptoms with meals, including nausea and diarrhea. He began to experience severe hypoglycemia events, including multiple episodes of loss of consciousness with requirement for emergency medical assistance. He was prescribed acarbose (with little improvement to his hypoglycemia) and a worsening of gastrointestinal symptoms. The patient was referred for trial participation.

In contrast to Patient 1, Patient 2 maintained a stable body weight throughout Baseline, Treatment Period 1 and Treatment Period 2 (−0.8% change). He also did not demonstrate dietary liberalization (as instructed per protocol to maintain a consistent diet throughout). However, his CNAQ, SHS-GI, and HFS-II scores showed notable improvement across domains, demonstrating that improved appetite/hunger and taste, reduced gastrointestinal symptoms, and reduced hypoglycemia-associated worries was capable of enabling (had the protocol allowed for dietary liberalization) safe and well-tolerated dietary liberalization, with improvement in the patient's nutritional status.

Among other things, Patient 2 differed from Patient 1 in that Patient 2 had over 20 severe hypoglycemia events at baseline and none on treatment. Over time, and if off of protocol, Patient 2 is expected to experience healthy weight regain due to prevention of hypoglycemic events, improved appetite, reduced GI symptoms, etc. Another difference between the two patients was that Patient 1 was in early post-operative period (within 6 months of surgery), whereas Patient 2 was latent (about 2.75 after surgery). The above-discussed study demonstrated, among other things, the ability of the methods according to the present disclosure to treat early and involuntary weight loss or effect weight regain in patients who has recently undergone gastrectomy.

### Example 5. Animal Study Demonstrating Increased Feeding Behaviors Upon Avexitide Administration

### A. Overview:

A an animal study involving subcutaneous administration of avexitide to healthy Sprague Dawley rats wase performed. Study results, which are described below, indicate that GLP1R antagonism may prevent unwanted weight loss in human subjects by increasing feeding behaviors and thereby aid in caloric intake and/or nutritional status. Currently existing body of preclinical and clinical data (some of which discussed elsewhere in the present disclosure) has shown that GLP1R agonism leads to weight loss and taste aversion via central mechanisms, but that GLP1R antagonism does not stimulate appetite, increase feeding behaviors, or cause weight gain. In contrast, the current study demonstrated that administration of GLP1R antagonist avexitide increased food and caloric consumption in study subjects. The study also demonstrated that administration of GLP1R antagonist avexitide increased feeding behaviors without causing meaningful weight gain, suggesting that avexitide may prevent unwanted weight loss and improve nutritional status.

### B. Study Description:

The objective of the study was to test for the effects of twice daily (BID) subcutaneous (SC) administration of avexitide dosing of CD® Sprague Dawley IGS (Crl:CD[SD]) on male and female rats before cohabitation, through mating, and to embryo implantation. The study design is summarized in Table 7. Among other parameters, the data on body weight and food consumption were collected throughout the study.

TABLE 7
Animal study design.

| | | Dose | Dose | Dose | Dose | No. of Animals | |
|---|---|---|---|---|---|---|---|
| Group | Test | Level^{a} | Level | Volume^{b} | Concentration | Main Study | |
| No. | Material | (mg/kg/day) | (mg/kg/dose) | (mL/kg/dose) | (mg/mL) | Males | Females |
| 1 | Reference | 0 | 0 | 5 | 0 | 22 | 22 |
| | Item^{c} | | | | | | |
| 2 | Avexitide | 45 | 22.5 | 5 | 4.5 | 22 | 22 |
| 3 | Avexitide | 150 | 75 | 5 | 15 | 22 | 22 |
| 4 | Avexitide | 450 | 225 | 5 | 45 | 22 | 22 |

^{a}Animals were dosed by subcutaneous bolus injection, twice daily. Males were dosed 28 days before cohabitation, during cohabitation, and continuing through the day before euthanasia. Females were dosed 15 days before cohabitation, during cohabitation and continuing until Gestation Day 7. Females without evidence of mating after completion of the cohabitation period were dosed until the day before necropsy.
^{b}Based on the most recent body weight measurement.
^{c}Acetate buffer with mannitol in sterile water for injection, USP, pH 5.5 ± 0.2.

### C. Results:

No avexitide-related effects on body weight, body weight gain, or organ weights were observed. However, increased food consumption was observed in males in a dose-dependent manner, when compared to controls, as follows: on Days 4 to 8 at all dose levels; on Days-1 to 4 and 18 to 22 at dose levels 150 and 450 mg/kg/day; and on Days 11 to 15 at dose level 450 mg/kg/day. In females, during the dosing period prior to mating, increased food consumption was observed in a dose-dependent manner as follows, when compared to controls: on Days 4 to 8 at dose level 450 mg/kg/day; on Days 8 to 11 at dose levels 150 and 450 mg/kg/day, and on Days 11 to 15 at dose levels 45 and 450 mg/kg/day. During gestation, increased food consumption was observed in female rats, when compared to controls, in non-dose-dependent manner, on Gestation Days (GDs) 0 to 3 at dose level 450 mg/kg/day, and on GDs 3 to 7 and to 8 at dose levels 150 and 450 mg/kg/day. The results of the study are summarized in Tables 8-10.

TABLE 8
Daily food consumption in males (g/animal/day).

| | | Day(s) Relative to Start Date | | | | | | | | | |
|---|---|---|---|---|---|---|---|---|---|---|---|
| | | | | | | | | | | | overall |
| | | −4-−1 | −1-4 | 4-8 | 8-11 | 11-15 | 15-18 | 18-22 | 22-25 | 25-28 | −1-28 |
| Group | | [G] | [G] | [G] | [G1] | [G1] | [G1] | [G1] | [G1] | [G1] | [G1] |
| 1 | Mean | 25.47 | 25.64 | 25.49 | 29.02 | 26.75 | 27.29 | 27.14 | 29.98 | 28.65 | 27.35 |
| | SD | 0.82 | 1.30 | 1.54 | 2.01 | 1.74 | 1.99 | 1.89 | 1.56 | 3.48 | 1.87 |
| | N | 22 | 22 | 22 | 22 | 22 | 22 | 22 | 22 | 16 | 16 |
| 2 | Mean | 25.71 | 27.10 | 27.00 * | 28.76 | 27.10 | 27.21 | 27.97 | 28.39 | 27.62 | 27.59 |
| | SD | 1.09 | 1.41 | 1.61 | 2.20 | 2.10 | 1.71 | 1.81 | 1.58 | 1.90 | 1.57 |
| | N | 22 | 22 | 22 | 22 | 22 | 22 | 22 | 22 | 22 | 22 |
| | % Diff | 0.95 | 5.72 | 5.93 | −0.89 | 1.32 | −0.28 | 3.06 | −5.31 | −3.58 | 0.90 |
| 3 | Mean | 25.89 | 28.85 ** | 27.63 ** | 29.25 | 28.06 | 27.88 | 28.80 * | 29.80 | 29.33 | 28.63 |
| | SD | 2.17 | 2.95 | 3.06 | 3.14 | 3.33 | 2.92 | 2.76 | 2.85 | 3.07 | 2.87 |
| | N | 22 | 22 | 22 | 20 | 22 | 22 | 22 | 22 | 22 | 22 |
| | % Diff | 1.67 | 12.54 | 8.38 | 0.81 | 4.89 | 2.17 | 6.11 | −0.61 | 2.40 | 4.69 |
| 4 | Mean | 25.45 | 29.60 ** | 28.36 ** | 29.12 | 28.51 * | 28.48 | 28.70 * | 29.24 | 28.82 | 28.85 |
| | SD | 0.75 | 1.82 | 1.54 | 1.49 | 1.68 | 1.64 | 1.97 | 2.15 | 2.06 | 1.52 |
| | N | 22 | 22 | 22 | 22 | 22 | 22 | 22 | 22 | 22 | 22 |
| | % Diff | −0.06 | 15.47 | 11.28 | 0.37 | 6.58 | 4.39 | 5.78 | −2.48 | 0.60 | 5.48 |

[G] - Kruskal-Wallis & Dunn: * = p ≤ 0.05; ** = p ≤ 0.01
[G1] - Anova & Dunnett: * = p ≤ 0.05

TABLE 9
Daily food consumption in females (g/animal/day).

| | | Day(s) Relative to Start Date | | | | | | | | | |
|---|---|---|---|---|---|---|---|---|---|---|---|
| | | | | | | | | | | | overall |
| | | −14-11 | −11-−7 | −7-−4 | −4-−1 | 1-4 | −1-4 | 4-8 | 8-11 | 11-15 | −1-15 |
| Group | | [G] | [G] | [G1] | [G] | [I] | [G] | [G] | [G] | [G1] | [G] |
| 1 | Mean | 17.65 | 18.52 | 18.88 | 17.07 | | 18.95 | 18.35 | 20.52 | 18.48 | 18.99 |
| | SD | 1.55 | 1.47 | 1.08 | 3.73 | | 1.63 | 1.25 | 1.61 | 1.11 | 1.23 |
| | N | 22 | 22 | 22 | 22 | | 22 | 22 | 22 | 20 | 20 |
| 2 | Mean | 17.75 | 18.38 | 19.19 | 18.30 | 17.67 n | 18.73 | 18.56 | 19.42 | 20.26 * | 19.29 |
| | SD | 1.45 | 1.63 | 1.22 | 2.02 | 0.00 | 1.70 | 1.95 | 1.78 | 3.53 | 1.72 |
| | N | 22 | 22 | 22 | 22 | 2 | 20 | 22 | 12 | 22 | 22 |
| | % Diff | 0.54 | −0.78 | 1.63 | 7.25 | . | −1.21 | 1.11 | −5.35 | 9.67 | 1.57 |
| 3 | Mean | 17.21 | 18.17 | 19.35 | 17.30 | . | 18.90 | 19.36 | 21.88 * | 19.32 | 19.73 |
| | SD | 0.90 | 1.44 | 1.96 | 3.69 | . | 1.02 | 1.42 | 1.11 | 1.68 | 0.97 |
| | N | 22 | 22 | 22 | 22 | . | 22 | 22 | 22 | 22 | 22 |
| | % Diff | −2.53 | −1.90 | 2.49 | 1.38 | . | −0.30 | 5.51 | 6.65 | 4.56 | 3.88 |
| 4 | Mean | 17.37 | 18.53 | 19.05 | 17.93 | 21.00 n | 19.75 | 19.52 * | 21.79 * | 19.47 * | 20.05 |
| | SD | 1.03 | 1.77 | 1.07 | 3.64 | 0.00 | 2.93 | 0.91 | 1.58 | 0.84 | 1.25 |
| | N | 22 | 22 | 20 | 22 | 2 | 20 | 22 | 22 | 22 | 22 |
| | % Diff | −1.59 | 0.06 | 0.91 | 5.06 | . | 4.20 | 6.38 | 6.20 | 5.36 | 5.55 |

[G] - Anova & Dunnett: * = p ≤ 0.05
[G1] - Kruskal-Wallis & Dunn
[I] - n - Inappropriate for statistics

TABLE 10
Daily food consumption in females during gestation (g/animal/day).

| | | Day(s) Relative to Mating | | | | | | |
|---|---|---|---|---|---|---|---|---|
| | | 0-3 | 3-7 | 7-8 | 8-10 | 10-13 | 0-8 | 8-13 |
| Group | | [G] | [G] | [G] | [G] | [G1] | [G] | [G] |
| 1 | Mean | 19.65 | 20.65 | 21.27 | 21.29 | 22.22 | 20.35 | 21.85 |
| | SD | 0.99 | 1.52 | 1.94 | 1.00 | 1.21 | 1.01 | 0.94 |
| | N | 18 | 18 | 18 | 18 | 18 | 18 | 18 |
| 2 | Mean | 20.21 | 22.01 | 20.55 | 21.59 | 21.94 | 21.15 | 21.80 |
| | SD | 2.76 | 2.66 | 7.04 | 1.84 | 1.64 | 2.85 | 1.62 |
| | N | 22 | 22 | 22 | 22 | 22 | 22 | 22 |
| | % Diff | 2.89 | 6.61 | −3.40 | 1.41 | −1.27 | 3.96 | −0.23 |
| 3 | Mean | 20.79 | 24.20 ** | 24.56 ** | 21.92 | 21.64 | 22.94 ** | 21.75 |
| | SD | 1.44 | 2.20 | 2.89 | 1.56 | 1.46 | 1.70 | 1.36 |
| | N | 20 | 20 | 18 | 20 | 20 | 18 | 20 |
| | % Diff | 5.82 | 17.24 | 15.45 | 2.94 | −2.60 | 12.75 | −0.44 |
| 4 | Mean | 21.71 ** | 23.90 ** | 24.36 ** | 21.25 | 23.14 | 23.14 ** | 22.38 |
| | SD | 1.75 | 1.54 | 1.99 | 2.23 | 1.94 | 1.53 | 1.90 |
| | N | 22 | 22 | 22 | 22 | 22 | 22 | 22 |
| | % Diff | 10.52 | 15.75 | 14.55 | −0.20 | 4.11 | 13.70 | 2.43 |

[G] - Kruskal-Wallis & Dunn: ** = p ≤ 0.01
[G1] - Anova & Dunnett: * = p ≤ 0.05

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes. No admission is made that any reference, including any non-patent or patent document cited in this specification, constitutes prior art. In particular, it will be understood that, unless otherwise stated, reference to any document herein does not constitute an admission that any of these documents form part of the common general knowledge in the art in the United States or in any other country. Any discussion of the references states what their authors assert, and the applicant reserves the right to challenge the accuracy and pertinence of any of the documents cited herein. The present disclosure shall control in the event there are any disparities between any definitions and/or description found in the cited references.

### PUBLICATIONS CITED IN THE DISCLOSURE

1. Brubaker et al. “Structure-function of the glucagon receptor family of G protein-coupled receptors: the glucagon, GIP, GLP-1, and GLP-2 receptors” Receptors & Channels. 8 (3-4): 179-88 (2002) doi:10.1080/10606820213687
2. Holst “The Physiology of Glucagon-like Peptide 1” Physiol. Rev. 87:1409-1439 (2007) doi:10.1152/physrev.00034.2006
3. Kittah et al. “A Pilot Study Examining the Effects of GLP-1 Receptor Blockade Using Exendin-(9,39) on Gastric Emptying and Caloric Intake in Subjects With and Without Bariatric Surgery.” Metab Syndr Relat Disord. 18(9):406-412 (2020). doi:10.1089/met.2020.0049.
4. Kulve et al. “Elevated Postoperative Endogenous GLP-1 Levels Mediate Effects of Roux-en-Y Gastric Bypass on Neural Responsivity to Food Cues.” Diabetes Care. 40(11):1522-1529 (2017). doi:10.2337/dc16-2113.
5. Landi et al. “Anorexia, physical function, and incident disability among the frail elderly population: results from the ilSIRENTE study.” J Am Med Dir Assoc. 11(4):268-274 (2010). doi:10.1016/j.jamda.2009.12.088
6. Martin et al. Ann N Y Acad Sci “Modulation of taste sensitivity by GLP-1 signaling in taste buds.” Ann N Y Acad Sci. 1170:98-101 (2009) doi:10.1111/j.1749-6632.2009.03920.x.
7. Maurer et al. Chapter 11 “Gastric emptying scintigraphy,” Gastroparesis. Edited by McCallum, and Parkman, Academic Press (2021), pages 125-142. doi:10.1016/B978-0-12-818586-5.00011-9
8. Melhorn et al. Appetite “Initial evidence that GLP-1 receptor blockade fails to suppress postprandial satiety or promote food intake in humans.” Appetite. (2014) 82:85-90. doi:10.1016/j.appet.2014.07.009.
9. Montrose-Rafizadeh et al. “High potency antagonists of the pancreatic glucagon-like peptide-1 receptor” Journal of Biological Chemistry. 272:21201-21206 (1997) doi:10.1074/jbc.272.34.21201
10. Müller et al. “Glucagon-like peptide 1 (GLP-1).” Mol Metab. 30:72-130 (2019). doi:10.1016/j.molmet.2019.09.010.
11. Salehi et al. “Blockade of glucagon-like peptide 1 receptor corrects postprandial hypoglycemia after gastric bypass.” Gastroenterology. 146(3):669-680.e2 (2014). doi:10.1053/j.gastro.2013.11.044.
12. Shah et al. “Contribution of endogenous glucagon-like peptide 1 to glucose metabolism after Roux-en-Y gastric bypass.” Diabetes 63:483-493 (2014).
13. Sisley et al. “Neuronal GLP1R mediates liraglutide's anorectic but not glucose-lowering effect.” J Clin Invest. 124(6):2456-63 (2014). doi:10.1172/JCI72434.
14. Wilson et al. “Appetite assessment: simple appetite questionnaire predicts weight loss in community-dwelling adults and nursing home residents.” Am J Clin Nutr. 82(5):1074-1081 (2005). doi:10.1093/ajcn/82.5.1074.

What is claimed is:

1. A method of decreasing the rate of hypoglycemia events in a subject,
wherein the method comprises administering a pharmaceutical formulation comprising avexitide or a pharmaceutically acceptable salt thereof,
wherein the pharmaceutical formulation is administered once daily (QD) and the avexitide in the pharmaceutical formulation is at a dose of about 20 mg to about 120 mg,
wherein the pharmaceutical formulation comprises avexitide at a concentration between about 2 mg/mL and about 250 mg/mL,
wherein the subject has previously undergone a surgery selected from the group consisting of Roux-en-Y gastric bypass, vertical sleeve gastrectomy, gastrectomy, esophagectomy, and Nissen fundoplication, and
wherein the rate of hypoglycemia events in the subject is reduced by at least about 10% relative to baseline.

2. The method of claim 1, wherein prior to administering the pharmaceutical formulation, the subject had elevated levels of glucagon-like peptide 1 (GLP-1) in comparison to a reference population level.

3. The method of claim 1, wherein the pharmaceutical formulation is administered subcutaneously.

4. The method of claim 1, wherein the pharmaceutical formulation is a buffered liquid formulation.

5. The method of claim 4, wherein the buffered liquid formulation comprises sodium acetate.

6. The method of claim 5, wherein the buffered liquid formulation comprises sodium acetate at a concentration of about 10 mM.

7. The method of claim 1, wherein the avexitide or the pharmaceutically acceptable salt thereof is at a concentration of about 100 mg/mL.

8. The method of claim 4, wherein the pH of the pharmaceutical formulation is about 5.1 to about 6.

9. The method of claim 8, wherein the pH of the pharmaceutical formulation is about 5.5.

10. The method of claim 1, wherein the pharmaceutical formulation comprises a tonicity modifier.

11. The method of claim 10, wherein the tonicity modifier is mannitol.

12. The method of claim 11, wherein mannitol is present in an amount from about 20 mg/mL to about 75 mg/mL.

13. The method of claim 1, wherein the method comprises administering to the subject the pharmaceutical formulation at a dose of 90 mg of avexitide QD for a first period of 14 days.

14. The method of claim 13, wherein the rate of hypoglycemia events in the subject is reduced by at least about 20%, about 30%, or about 40% relative to baseline.

15. The method of claim 13, wherein the rates of Level 1 hypoglycemia events, Level 2 hypoglycemia events, and Level 3 hypoglycemia events are reduced relative to baseline.

16. The method of claim 15, wherein:
a Level 1 hypoglycemia event is defined as a self-monitoring of blood glucose (SMBG) level of <70 mg/dL,
a Level 2 hypoglycemia event is defined as an SMBG level of <54 mg/dL, and
a Level 3 hypoglycemia event is defined as an event characterized by altered mental and/or physical functioning of the subject.

17. The method of claim 15, wherein the rate of Level 1 hypoglycemia events is reduced by about 68% relative to baseline.

18. The method of claim 15, wherein the rate of Level 2 hypoglycemia events is reduced by about 53% relative to baseline.

19. The method of claim 15, wherein the rate of Level 3 hypoglycemia events is reduced by about 66% relative to baseline.

20. The method of claim 15, wherein the mean percent time spent with a glucose level of <54 mg/dL is reduced by about 64% relative to baseline.

21. The method of claim 13, wherein the method further comprises administering to the subject the pharmaceutical formulation at a dose of 45 mg of avexitide twice daily (BID) for a second period of 14 days.
